(19) United States
(12) Patent Application Publication
(10) Pub. No.: US 2026/0369089 A1
(43) Pub. Date: Nov. 19, 2026

(54) INFORMATION PROCESSING DEVICE, BIOLOGICAL SAMPLE ANALYSIS SYSTEM, AND BIOLOGICAL SAMPLE ANALYSIS METHOD

(71) Applicant: SONY GROUP CORPORATION, TOKYO (JP)

(72) Inventors: NOE SATO, TOKYO (JP); KAZUHIRO NAKAGAWA, TOKYO (JP); KENJI IKEDA, TOKYO (JP); TOMOHIKO NAKAMURA, TOKYO (JP); TETSURO KUWAYAMA, TOKYO (JP)

(21) Appl. No.: 19/155,269

(22) PCT Filed: Feb. 15, 2024
(86) PCT No.: PCT/JP2024/005172

§ 371 Date: Aug. 11, 2025

(30) Foreign Application Priority Data
Mar. 3, 2023 (JP) 2023-033049

(51) Int. Cl. G06V 20/69; G06V 10/25; G06V 10/762

(52) U.S. Cl. CPC G06V 20/698; G06V 10/25; G06V 10/762; G06V 2201/03

(57) ABSTRACT

An information processing device according to an aspect of the present disclosure includes an acquisition unit that acquires, from a sample including a biological sample, a fluorescence spectrum deriving from the biological sample and positional information of the biological sample, a specifying unit that specifies, from the fluorescence spectrum, pieces of information concerning a plurality of different biomarkers of the biological sample linked with the positional information of the biological sample, a standardization unit that executes information complementation or exclusion on the pieces of information concerning the plurality of biomarkers, and a correlation analysis unit that performs, on the pieces of information concerning the plurality of biomarkers for which the information complementation or exclusion has been performed, matrix decomposition processing corresponding to a combination of the plurality of biomarkers and outputs a correlation among the pieces of information concerning the plurality of biomarkers.

### FIELD

The present disclosure relates to an information processing device, a biological sample analysis system, and a biological sample analysis method.

### BACKGROUND

Currently, as illustrated in the process of the cancer immune cycle, it is considered that an interaction between cells (for example, contact between cells) triggers the progression or eradication of cancer. However, it is difficult to algorithmize a relationship of the interaction between cells. For example, it is difficult to obtain an algorithm that satisfies a biological aspect. In order to grasp the interaction between cells, it is important to carry out a correlation analysis between biomarkers. There is a clustering method for clustering a plurality of biomarkers. For example, Patent Literature 1 proposes a clustering method using NMF (Non-negative Matrix Factorization).

### CITATION LIST

### Patent Literature

Patent Literature 1: JP 2016-31678 A

### SUMMARY

### Technical Problem

However, for example, in pathological image clustering using joint non-negative matrix factorization (jNMF) or the like, it is necessary to align the number of blocks in a field of view, that is, the number of pieces of data in a matrix (a matrix size) for each of specimens, which are samples. However, since the number of pieces of data in the matrix (for example, in the row direction) is different for each of the specimens, the number of pieces of data in the matrix (for example, the column direction) obtained for each of the specimens by clustering is sometimes different. That is, the clustering tends to be insufficient and it is difficult to accurately obtain a correlation among the plurality of biomarkers.

Therefore, the present disclosure proposes an information processing device, a biological sample analysis system, and a biological sample analysis method capable of accurately obtaining a correlation among a plurality of biomarkers.

### Solution to Problem

An information processing device according to an aspect of the present disclosure includes: an acquisition unit that acquires, from a sample including a biological sample, a fluorescence spectrum deriving from the biological sample and positional information of the biological sample; a specifying unit that specifies, from the fluorescence spectrum, pieces of information concerning a plurality of different biomarkers of the biological sample linked with the positional information of the biological sample; a standardization unit that executes information complementation or exclusion on the pieces of information concerning the plurality of biomarkers; and a correlation analysis unit that performs, on the pieces of information concerning the plurality of biomarkers for which the information complementation or exclusion has been performed, matrix decomposition processing corresponding to a combination of the plurality of biomarkers and outputs a correlation among the pieces of information concerning the plurality of biomarkers.

A biological sample analysis system according to an aspect of the present disclosure includes: an imaging device that acquires a specimen image of a sample including a biological sample; and an information processing device that processes the specimen image, wherein the information processing device includes: an acquisition unit that acquires, from the specimen image, a fluorescence spectrum deriving from the biological sample and positional information of the biological sample; a specifying unit that specifies, from the fluorescence spectrum, pieces of information concerning a plurality of different biomarkers of the biological sample linked with the positional information of the biological sample; a standardization unit that executes information complementation or exclusion on the pieces of information concerning the plurality of biomarkers; and a correlation analysis unit that performs, on the pieces of information concerning the plurality of biomarkers for which the information complementation or exclusion has been performed, matrix decomposition processing corresponding to a combination of the plurality of biomarkers and outputs a correlation among the pieces of information concerning the plurality of biomarkers.

A biological sample analysis method according to an aspect of the present disclosure includes: acquiring, from a sample including a biological sample, a fluorescence spectrum deriving from the biological sample and positional information of the biological sample; specifying, from the fluorescence spectrum, pieces of information concerning a plurality of different biomarkers of the biological sample linked with the positional information of the biological sample; executing information complementation or exclusion on the pieces of information concerning the plurality of biomarkers; and performing, on the pieces of information concerning the plurality of biomarkers for which the information complementation or exclusion has been performed, matrix decomposition processing corresponding to a combination of the plurality of biomarkers and outputting a correlation among the pieces of information concerning the plurality of biomarkers.

### BRIEF DESCRIPTION OF DRAWINGS

FIG. 1 is a diagram illustrating an example of a schematic configuration of an information processing system according to an embodiment of the present disclosure.

FIG. 2 is a flowchart illustrating an example of an overall processing flow by an information processing device according to the embodiment of the present disclosure.

FIG. 3 is a diagram illustrating an example of a schematic configuration of a space analysis unit according to the embodiment of the present disclosure.

FIG. 4 is a flowchart illustrating an example of a flow of correlation analysis processing for multi-biomarkers according to the embodiment of the present disclosure.

FIG. 5 is a diagram for explaining an example of a sample according to the embodiment of the present disclosure.

FIG. 6 is a diagram illustrating an example of a positive cell rate for each of blocks of AF488_CD7 according to the embodiment of the present disclosure.

FIG. 7 is a diagram illustrating an example of a positive cell rate for each of blocks of AF555_CD3 according to the embodiment of the present disclosure.

FIG. 8 is a diagram illustrating an example of a positive cell rate for each of blocks after sorting of AF488_CD7 according to the embodiment of the present disclosure.

FIG. 9 is a diagram illustrating an example of a positive cell rate for each of blocks after sorting of AF647_CD5 according to the embodiment of the present disclosure.

FIG. 10 is a diagram for explaining an example of jNMF according to the embodiment of the present disclosure.

FIG. 11 is a flowchart illustrating an example of a flow of standardization processing for the number of pieces of data according to the embodiment of the present disclosure.

FIG. 12 is a diagram for explaining an example of jNMF results in a data set without complementation according to the embodiment of the present disclosure.

FIG. 13 is a diagram for explaining an example of jNMF results in a data set 1 complemented by a data complementation method of a comparative example according to the embodiment of the present disclosure.

FIG. 14 is a diagram for explaining an example of data complementation processing according to the embodiment of the present disclosure.

FIG. 15 is a flowchart illustrating an example of a flow of data complementation processing according to the embodiment of the present disclosure.

FIG. 16 is a diagram for explaining an example of jNMF results in the data set 1 complemented by the data complementation method according to the embodiment of the present disclosure.

FIG. 17 is a diagram for explaining an example of jNMF results in a data set 2 complemented by the data complementation method according to the embodiment of the present disclosure.

FIG. 18 is a diagram illustrating a plurality of types of markers according to the embodiment of the present disclosure.

FIG. 19 is a diagram for explaining an example of blocks of a sample according to the embodiment of the present disclosure.

FIG. 20 is a flowchart illustrating an example of a flow of data removal processing according to the embodiment of the present disclosure.

FIG. 21 is a diagram for explaining an example of presenting a Sample 12 subjected to data complementation to a user according to the embodiment of the present disclosure.

FIG. 22 is a flowchart illustrating an example of a flow of coincidence degree calculation processing according to the embodiment of the present disclosure.

FIG. 23 is a diagram for explaining an example of a display image according to the embodiment of the present disclosure.

FIG. 24 is a diagram for explaining an example of a display image according to the embodiment of the present disclosure.

FIG. 25 is a diagram for explaining an example of a display image according to the embodiment of o the present disclosure.

FIG. 26 is a diagram for explaining an example of a display image according to the embodiment of the present disclosure.

FIG. 27 is a diagram for explaining an example of a display image according to the embodiment of the present disclosure.

FIG. 28 is a diagram for explaining an example of a display image according to the embodiment of the present disclosure.

FIG. 29 is a diagram for explaining an example of a display image according to the embodiment of the present disclosure.

FIG. 30 is a diagram for explaining an example of a display image according to the embodiment of the present disclosure.

FIG. 31 is a diagram for explaining an example of a display image according to the embodiment of the present disclosure.

FIG. 32 is a flowchart illustrating an example of a flow of data complementation processing according to an embodiment of the present disclosure.

FIG. 33 is a diagram for explaining an example of data complementation processing according to the embodiment of the present disclosure.

FIG. 34 is a diagram for explaining an example of data complementation processing according to the embodiment of the present disclosure.

FIG. 35 is a diagram illustrating an example of a schematic configuration of a fluorescence observation device.

FIG. 36 is a diagram illustrating an example of a schematic configuration of an observation unit.

FIG. 37 is a diagram illustrating an example of a sample.

FIG. 38 is an enlarged view illustrating a region where a sample is irradiated with line illumination.

FIG. 39 is a diagram schematically illustrating an overall configuration of a microscope system.

FIG. 40 is a diagram illustrating an example of an imaging scheme.

FIG. 41 is a diagram illustrating an example of an imaging scheme.

FIG. 42 is a diagram illustrating an example of a schematic configuration of hardware of the information processing device.

### DESCRIPTION OF EMBODIMENTS

Hereinafter, embodiments (including examples and modifications) of the present disclosure are explained in detail with reference to the drawings. Note that a device, a system, a method, and the like according to the present disclosure are not limited by the embodiments of the present disclosure. In the embodiments explained below, redundant explanation is omitted by adding the same reference signs to basically the same parts.

One or a plurality of embodiments (including examples and modifications) explained below can be respectively independently implemented. On the other hand, at least a part of the plurality of embodiments explained below may be implemented in combination with at least a part of other embodiments as appropriate. These plurality of embodiments can include new characteristics different from one another. Therefore, these plurality of embodiments can contribute to solving objects or problems different from one another and can achieve effects different from one another.

The present disclosure is explained according to the order of items described below.
1. Embodiment
1-1. Configuration example of an information processing system
1-2. Processing example of an information processing device
1-3. Processing example of a correlation analysis of multi-biomarkers
1-4. Specific examples of the correlation analysis of the multi-biomarkers
1-5. Standardization method for the number of pieces of data
1-5-1. Processing example of standardization of the number of pieces of data
1-5-2. Data complementation method
1-5-3. Data exclusion method
1-6. Coincidence degree calculation method
1-7. Display method
1-7-1. Display example of a tissue image of a sample and a common module
1-7-2. Display example of jNMF results
1-8. Other examples of a data complementation method
1-9. Action and effects
2. Other embodiments
3. Applied examples
4. Application examples
5. Configuration example of hardware
6. Appendixes

### 1. EMBODIMENT

### 1-1. Configuration Example of an Information Processing System

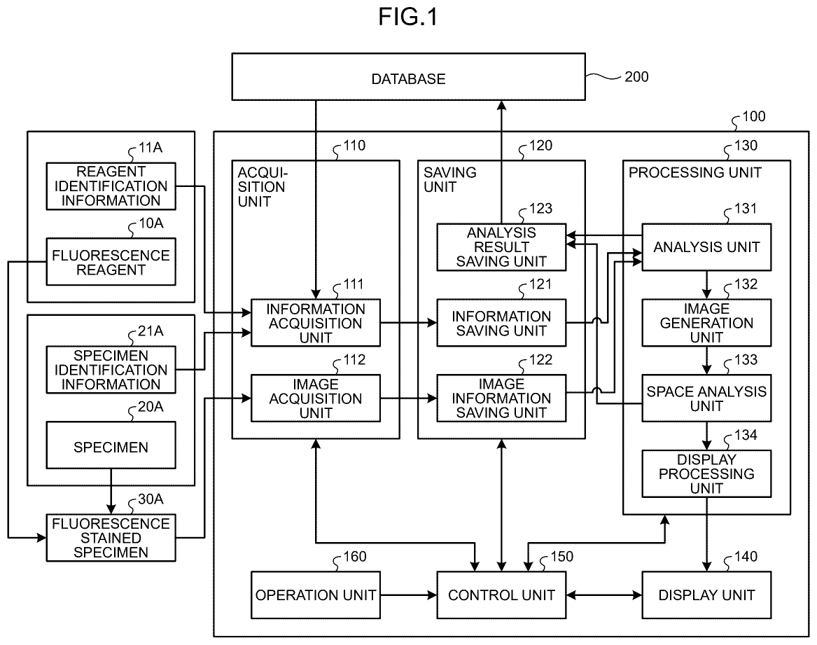

A configuration example of an information processing system according to the present embodiment is explained with reference to FIG. 1. FIG. 1 is a diagram illustrating an example of a schematic configuration of the information processing system according to the present embodiment. The information processing system is an example of a biological sample analysis system.

As illustrated in FIG. 1, the information processing system according to the present embodiment includes an information processing device 100 and a database 200. As inputs to the information processing system, a fluorescence reagent 10A, a specimen 20A, and a fluorescence stained specimen 30A are present.

### Fluorescence Reagent 10A

The fluorescence reagent 10A is a chemical used for staining the specimen 20A. The fluorescence reagent 10A is, for example, a fluorescent antibody (a primary antibody used for direct labeling or a secondary antibody used for indirect labeling), a fluorescent probe, a nuclear staining reagent, or the like. However, types of the fluorescence reagent 10A are not particularly limited these. The fluorescence reagent 10A is managed with identification information (hereinafter referred to as “reagent identification information 11A”) capable of identifying the fluorescence reagent 10A (and a production lot of the fluorescence reagent 10A). The reagent identification information 11A is, for example, bar code information (one-dimensional bar code information, two-dimensional bar code information, or the like) but is not limited to the barcode information. Properties of the fluorescence reagent 10A are different for each of production lots depending on a production method, a state of cells from which an antibody is acquired, and the like even for the same (same type) product. For example, in the fluorescence reagent 10A, spectral information, a quantum yield, a fluorescent labeling rate (also referred to as “F/P value: Fluorescein/Protein”, which indicates the number of fluorescent molecules labeling an antibody), or the like is different for each of the production lots. Thus, in the information processing system according to the present embodiment, the fluorescence reagent 10A is managed for each of the production lots with the reagent identification information 11A added thereto (in other words, reagent information of fluorescence reagents 10A is managed for each of the production lots). Accordingly, the information processing device 100 can separate the fluorescence signal and the autofluorescence signal considering a slight difference in properties that appears for each of the production lots. Note that the fluorescence reagent 10A being managed in units of production lots is only an example. The fluorescence reagent 10A may be managed in units finer than the production lots.

### Specimen 20A

The specimen 20A is prepared for the purpose of a pathological diagnosis, a clinical examination, or the like from a sample or a tissue sample collected from a human body. For the specimen 20A, a type of a tissue (for example, an organ or a cell) to be used, a type of a disease to be targeted, an attribute (for example, an age, sex, a blood type, or a race) of a subject, or a lifestyle (for example, dietary habits, exercise habits, or smoking habits) of the subject is not particularly limited. The specimen 20A is managed with identification information capable of identifying the specimens 20A (hereinafter referred to as “specimen identification information 21A”) added thereto. Like the reagent identification information 11A, the specimen identification information 21A is, for example, bar code information (one-dimensional bar code information, two-dimensional bar code information, or the like) but is not limited to the barcode information or the like. A property of the specimen 20A is different depending on a type of a tissue to used, a type of a disease to be targeted, an attribute of a subject, a lifestyle of the subject, or the like. For example, in the specimen 20A, a measurement channel, spectral information, or the like is different depending on a type of a tissue to be used or the like. Thus, in the information processing system according to the present embodiment, the specimens 20A are individually managed by being added with the specimen identification information 21A. Accordingly, the information processing device 100 can separate a fluorescence signal and an autofluorescence signal, considering a slight difference in a property appearing for each of the specimens 20A.

### Fluorescence Stained Specimen 30A

The fluorescence stained specimen 30A is prepared by the specimen 20A being stained with the fluorescence reagent 10A. In the present embodiment, the fluorescence stained specimen 30A is assumed to be prepared by the specimen 20A being stained with at least one fluorescence reagent 10A. However, the number of fluorescence reagents 10A used for the staining is not particularly limited. A staining method is determined by, for example, a combination of each of the specimens 20A and each of the fluorescence reagents 10A and is not particularly limited. The fluorescence stained specimen 30A is input to the information processing device 100 and imaged.

### Information Processing Device 100

The information processing device 100 includes, as illustrated in FIG. 1, an acquisition unit 110, a saving unit 120, a processing unit 130, a display unit 140, a control unit 150, and an operation unit 160.

### Acquisition Unit 110

The acquisition unit 110 is configured to acquire information used for various types of processing of the information processing device 100. As illustrated in FIG. 1, the acquisition unit 110 includes an information acquisition unit 111 and an image acquisition unit 112.

### Information Acquisition Unit 111

The information acquisition unit 111 is configured to acquire various kinds of information such as reagent information and specimen information. More specifically, the information acquisition unit 111 acquires the reagent identification information 11A added to the fluorescence reagent 10A used for generating the fluorescence stained specimen 30A and the specimen identification information 21A added to the specimen 20A. For example, the information acquisition unit 111 acquires the reagent identification information 11A and the specimen identification information 21A using a barcode reader or the like. The information acquisition unit 111 acquires reagent information from the database 200 based on the reagent identification information 11A and acquires specimen information from the database 200 based on the specimen identification information 21A. The information acquisition unit 111 saves acquired these kinds of information in an information saving unit 121 explained below.

### Image Acquisition Unit 112

The image acquisition unit 112 is configured to acquire image information of the fluorescence stained specimen 30A (the specimen 20A stained with at least one fluorescence reagent 10A). More specifically, the image acquisition unit 112 includes any imaging element (for example, a CCD or a CMOS) and acquires image information by imaging the fluorescence stained specimen 30A using the imaging element. Here, note that the “image information” is a concept including not only an image itself of the fluorescence stained specimen 30A but also a measurement value that is not visualized as an image. For example, the image information may include information concerning a wavelength spectrum (hereinafter referred to as fluorescence spectrum) of fluorescence radiated from the fluorescence stained specimen 30A. The image acquisition unit 112 saves the image information in an image information saving unit 122 explained below.

### Saving Unit 120

The saving unit 120 is configured to save (store) information used for various processes of the information processing device 100 or information output by the various processes. As illustrated in FIG. 1, the saving unit 120 includes an information saving unit 121, an image information saving unit 122, and an analysis result saving unit 123.

### Information Saving Unit 121

The information saving unit 121 is configured to save various kinds of information such as reagent information and specimen information acquired by the information acquisition unit 111. Note that, after analysis processing by an analysis unit 131 explained below and generation processing for image information (reconstruction processing for image information) by an image generation unit 132 explained below are completed, the information saving unit 121 may increase a free space by deleting the reagent information and the specimen information used for the processing.

### Image Information Saving Unit 122

The image information saving unit 122 is configured to save image information of the fluorescence stained specimen 30A acquired by the image acquisition unit 112. Note that, like the information saving unit 121, after the analysis processing by the analysis unit 131 and the generation processing for the image information by the image generation unit 132 (the reconstruction processing for the image information) end, the image information saving unit 122 may increase a free space by deleting the image information used for the processing.

### Analysis Result Saving Unit 123

The analysis result saving unit 123 is configured to save a result of the analysis processing performed by an analysis unit 131 or a space analysis unit 133 explained below. For example, the analysis result saving unit 123 saves a fluorescence signal of the fluorescence reagent 10A or an autofluorescence signal of the specimen 20A separated by the analysis unit 131, a correlation analysis result or an effect prediction result (an effect estimation result) obtained by the space analysis unit 133, or the like. The analysis result saving unit 123 separately provides a result of the analysis processing to the database 200 in order to improve analysis accuracy through machine learning or the like. After providing the result of the analysis processing to the database 200, the analysis result saving unit 123 may increase a free space by deleting, as appropriate, the result of the analysis processing saved therein.

### Processing Unit 130

The processing unit 130 is a functional configuration for performing various types of processing using image information, reagent information, and specimen information. As illustrated in FIG. 1, the processing unit 130 includes an analysis unit 131, an image generation unit 132, a space analysis unit 133, and a display processing unit 134.

### Analysis Unit 131

The analysis unit 131 is configured to perform various kinds of analysis processing using image information, specimen information, and reagent information. For example, the analysis unit 131 performs processing (color separation processing) of separating an autofluorescence signal of the specimen 20A and a fluorescence signal of the fluorescence reagent 10A from the image information based on the specimen information and the reagent information.

Specifically, the analysis unit 131 recognizes one or more elements composing the autofluorescence signal based on a measurement channel included in the specimen information. For example, the analysis unit 131 recognizes one or more autofluorescence components composing the autofluorescence signal. The analysis unit 131 predicts the autofluorescence signal included in the image information using spectral information of these autofluorescence components included in the specimen information. The analysis unit 131 separates the autofluorescence signal and the fluorescence signal from the image information based on spectral information of a fluorescence component of the fluorescence reagent 10A included in the reagent information and the predicted autofluorescence signal.

Here, when the specimen 20A is stained with two or more fluorescence reagents 10A, the analysis unit 131 separates a fluorescence signal of each of these two or more fluorescence reagents 10A from the image information (or the fluorescence signal after being separated from the autofluorescence signal) based on the specimen information and the reagent information. For example, the analysis unit 131 separates the fluorescence signal of each of the fluorescence reagents 10A from the entire fluorescence signal after being separated from the autofluorescence signal using spectral information of fluorescence components of the fluorescence reagents 10A included in the reagent information.

When the autofluorescence signal is composed of two or more autofluorescence components, the analysis unit 131 separates an autofluorescence signal of each of autofluorescence components from the image information (or the autofluorescence signal after being separated from the fluorescence signal) based on the specimen information and the reagent information. For example, the analysis unit 131 separates the autofluorescence signal of each of the autofluorescence components from the entire autofluorescence signal after being separated from the fluorescence signal using the spectral information of the autofluorescence components included in the specimen information.

The analysis unit 131 that has separated the fluorescence signal and the autofluorescence signal performs various types of processing using these signals. For example, the analysis unit 131 may perform subtraction processing (also referred to as “background subtraction processing”) on image information of the other specimens 20A using the autofluorescence signal after the separation to extract fluorescence signals from the image information of the other specimens 20A. When there is a plurality of specimens 20A that are the same or similar in the viewpoints of a tissue used for the specimen 20A, a type of a disease to be targeted, an attribute of a subject, a lifestyle of the subject, and the like, it is highly likely that autofluorescence signals of these specimens 20A are similar. The similar specimens 20A referred to here include, for example, a tissue section before staining of a tissue section to be stained (hereinafter referred to as section), a section adjacent to a stained section, a section different from the stained section in the same block (sampled from the same place as the stained section), a section in a different block in the same tissue (sampled from a place different from the stained section), a section collected from a different patient, or the like. Thus, when an autofluorescence signal is successfully extracted from a certain specimen 20A, the analysis unit 131 may extract fluorescence signals from image information of the other specimens 20A by removing the autofluorescence signal from the image information of the other specimens 20A. When calculating an S/N value using the image information of the other specimens 20A, the analysis unit 131 can improve the S/N value by using a background after the removal of the autofluorescence signal.

Other than the background subtraction processing, the analysis unit 131 can perform various types of processing using the fluorescence signal or the autofluorescence signal after the separation. For example, the analysis unit 131 can analyze a fixation state of the specimen 20A using these signals and can perform segmentation (or region division) for recognizing a region of an object (for example, a cell, an intracellular structure (a cytoplasm, a cell membrane, a nucleus, or the like) or a tissue (a tumor site, a non-tumor site, a connective tissue, a blood vessel, a blood vessel wall, a lymphatic vessel, a fibrotic structure, necrosis, or the like)) included in the image information.

### Image Generation Unit 132

The image generation unit 132 is configured to generate image information based on an analysis result obtained by the analysis unit 131. The image generation unit 132 generates (recomposes) image information based on the fluorescence signal or the autofluorescence signal separated by the analysis unit 131. For example, the image generation unit 132 can generate image information including only the fluorescence signal or image information including only the autofluorescence signal. At that time, when the fluorescence signal is composed of a plurality of fluorescence components or the autofluorescence signal is composed of a plurality of autofluorescence components, the image generation unit 132 can generate image information in units of the respective components. Furthermore, when the analysis unit 131 performs various types of processing (for example, analysis of the fixation state of the specimen 20A, segmentation, or calculation of an S/N value) using the fluorescence signal or the autofluorescence signal after the separation, the image generation unit 132 may generate image information indicating results of those types of processing. According to this configuration, distribution information of the fluorescence reagents 10A labeled on target molecules or the like, that is, two-dimensional spread and intensity of fluorescence, a wavelength, and a positional relationship among the fluorescence reagents 10A are visualized. In particular, in a tissue image analysis region where information concerning a target substance is complicated, the visibility of a doctor or a researcher, who is a user, can be improved.

The image generation unit 132 may perform control to distinguish the fluorescence signal from the autofluorescence signal based on the fluorescence signal or the autofluorescence signal separated by the analysis unit 131 and generate image information. Specifically, the image generation unit 132 may generate the image information by controlling, for example, improving the luminance of a fluorescence spectrum of the fluorescence reagent 10A labeled on a target molecule or the like, extracting and discoloring of only the fluorescence spectrum of the labeled fluorescence reagent 10A, extracting fluorescence spectra of two or more fluorescence reagents 10A from the specimen 20A labeled with the two or more fluorescence reagents 10A and discoloring the fluorescent spectra into different colors, extracting and dividing or subtracting only an autofluorescence spectrum of the specimen 20A, and improving a dynamic range. Accordingly, the user can clearly distinguish color information deriving from a fluorescence reagent bound to the target substance and the visibility of the user can be improved.

### Space Analysis Unit 133

The space analysis unit 133 performs processing of analyzing a correlation among a plurality of biomarkers (for example, among tissues) from image information after color separation and processing of predicting a medicine effect based on a correlation analysis result that is an analysis result of the correlation. For example, the space analysis unit 133 performs a clustering analysis on a specimen image stained with a plurality of biomarkers while maintaining spatial information, that is, positional information to analyze the correlation among the biomarkers. Such correlation analysis processing and medicine effect prediction processing for multi-biomarkers are explained in detail below.

### Display Processing Unit 134

The display processing unit 134 generates image information including a correlation analysis result, an effect prediction result (an effect estimation result), and the like obtained by the space analysis unit 133 and transmits the image information to the display unit 140. This image information generation processing is explained in detail below. Note that the display processing unit 134 can directly transmit the image information generated by the image generation unit 132 to the display unit 140 or process the image information and transmit the image information to the display unit 140. For example, the display processing unit 134 is capable of performing processing of adding image information including the correlation analysis result, the effect prediction result, and the like obtained by the space analysis unit 133 to the image information generated by the image generation unit 132.

### Display Unit 140

The display unit 140 is configured to present the image information generated by the image generation unit 132, the display processing unit 134, and the like to the user by displaying the image information. Note that a type of the display used as the display unit 140 is not particularly limited. Although not explained in detail in the present embodiment, the image information generated by the image generation unit 132, the display processing unit 134, and the like may be presented to the user by being projected by a projector or printed by a printer (in other words, a method of outputting the image information is not particularly limited).

### Control Unit 150

The control unit 150 is a functional component that comprehensively controls overall processing performed by the information processing device 100. For example, the control unit 150 controls, based on operation input by the user performed via the operation unit 160, the start, the end, and the like of the various types of processing (for example, the imaging processing for the fluorescence stained specimen 30A, the analysis processing, the generation processing for image information (the reconstruction processing for image information), and the display processing for image information) explained above. Note that control content of the control unit 150 is not particularly limited. For example, the control unit 150 may control processing (for example, processing concerning an OS (Operating System)) generally performed in a general-purpose computer, a PC, a tablet PC, or the like.

### Operation Unit 160

The operation unit 160 is configured to receive operation input from the user. More specifically, the operation unit 160 includes various types of input means such as a keyboard, a mouse, buttons, a touch panel, or a microphone. The user can perform various kinds of input to the information processing device 100 by operating these input means. Information concerning the operation input performed via the operation unit 160 is provided to the control unit 150.

### Database 200

The database 200 is a device that manages specimen information, reagent information, and a result of analysis processing. More specifically, the database 200 manages the specimen identification information 21A and the specimen information and the reagent identification information 11A and the reagent information in linkage with each other. Accordingly, the information acquisition unit 111 can acquire the specimen information from the database 200 based on the specimen identification information 21A of the specimen 20A, which is a measurement target, and acquire the reagent information from the database 200 based on the reagent identification information 11A of the fluorescence reagent 10A. Note that the database 200 may manage the image information generated by the image generation unit 132, the display processing unit 134, and the like.

As explained above, the specimen information managed by the database 200 is information including the measurement channel and the spectral information specific to the autofluorescence component included in the specimen 20A. However, besides the measurement channel and the spectral information, the specimen information may include target information about the specimens 20A, specifically, information concerning a type of a tissue (for example, an organ, a cell, blood, body fluid, ascites, or pleural effusion) to be used, a type of a disease to be targeted, an attribute (for example, an age, sex, a blood type, or a race) of a subject, or a lifestyle (for example, dietary habits, exercise habits, or smoking habits) of the subject. Information including a measurement channel and spectral information specific to an autofluorescence component included in the specimen 20A target information may be linked with the specimen information for each of the specimens 20A. Accordingly, the information including the measurement channel and the spectral information specific to the autofluorescence component included in the specimen 20A can be easily traced from the target information. For example, it is possible to cause the analysis unit 131 to execute similar separation processing performed in the past from the similarity of the target information in the plurality of specimens 20A and reduce a measurement time. The “tissue to be used” is not particularly limited to a tissue collected from a target and may include an in vivo tissue or a cell line of a human, an animal, or the like, a solution, a solvent, a solute, and a material contained in a measurement target.

As explained above, the reagent information managed by the database 200 is the information including the spectral information of the fluorescence reagent 10A. However, besides the information, the reagent information may include information concerning the fluorescence reagent 10A such as a production lot, a fluorescent component, an antibody, a clone, a fluorescent labeling rate, a quantum yield, a fading coefficient (information indicating easiness of reducing fluorescence intensity of the fluorescence reagent 10A), and an absorption cross-sectional area (or a molar absorption coefficient). Furthermore, the specimen information and the reagent information managed by the database 200 may be managed in a different configuration. In particular, the information concerning the reagent may be a reagent database that presents an optimal combination of reagents to the user.

Here, it is assumed that the specimen information and the reagent information are provided from a producer (a manufacturer) or the like or are independently measured in the information processing system according to the present disclosure. For example, a producer of the fluorescence reagent 10A often does not measure and provide spectral information, a fluorescent labeling rate, and the like for each of production lots. Therefore, by independently measuring and managing these kinds of information in the information processing system according to the present disclosure, separation accuracy of the fluorescence signal and the autofluorescence signal can be improved. For simplification of management, the database 200 may use, as the specimen information and the reagent information (particularly, the reagent information), catalog values disclosed by a producer (a manufacturer) or the like, literature values described in various documents, or the like. However, in general, since actual specimen information and reagent information are often different from the catalog values and the literature values, it is more preferable that the specimen information and the reagent information be independently measured and managed in the information processing system according to the present disclosure as explained above.

The accuracy of the analysis processing (for example, the separation processing for the fluorescence signal and the autofluorescence signal, the correlation analysis processing for the multi-biomarkers, and the prediction processing for the medicine effect) can be improved by a machine learning technique or the like using the specimen information, the reagent information, and the result of the analysis processing managed in the database 200. An entity that performs learning using the machine learning technique or the like is not particularly limited. For example, the analysis unit 131 generates a classifier or an estimator subjected to machine learning with learning data using a neural network. Then, when various kinds of corresponding information are acquired anew, the analysis unit 131 inputs those kinds of information to the classifier or the estimator to perform the separation processing of the fluorescence signal and the autofluorescence signal, the correlation analysis processing for the multi-biomarkers, and the prediction processing for the medicine effect.

A method of obtaining similar processing performed in the past with higher accuracy than a predicted result, statistically or regressively analyzing processing content in those types of processing (information, parameters, and the like used for the processing), and improving the separation processing for the fluorescence signal and the autofluorescence signal, the correlation analysis processing for the multi-biomarkers, and the prediction processing for the medicine effect based on an analysis result may be output. Note that the machine learning method is not limited to the above and a publicly-known machine learning technique can be used. The separation processing for the fluorescence signal and the autofluorescence signal, the correlation analysis processing for the multi-biomarkers, and the prediction processing for the medicine effect may be performed by artificial intelligence. Other various types of processing (for example, analysis of the fixation state of the specimen 20A or segmentation) may be improved by a machine learning technique or the like.

The configuration example of the information processing system according to the present embodiment is explained above. Note that the configuration explained above with reference to FIG. 1 is only an example. The configuration of the information processing system according to the present embodiment is not limited to such an example. For example, the information processing device 100 may not always include all of the functional components illustrated in FIG. 1. The information processing device 100 may include the database 200 on the inside. The functional configuration of the information processing device 100 can be flexibly modified according to specifications and operations.

The information processing device 100 may perform processing other than the processing explained above. For example, the reagent information includes the information such as the quantum yield, the fluorescent labeling rate, and the absorption cross-sectional area (or the molar absorption coefficient) concerning the fluorescence reagent 10A, whereby the information processing device 100 may calculate the number of fluorescent molecules, the number of antibodies bound to the fluorescent molecules, and the like in the image information using the image information from which the autofluorescence signal has been removed and the reagent information.

### 1-2. Processing Example of the Information Processing Device

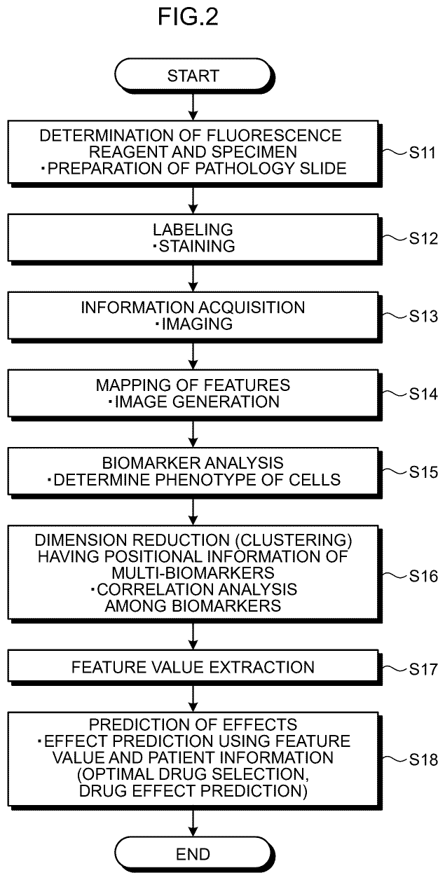

A processing example (an overall flow) of the information processing device 100 according to the present embodiment is explained with reference to FIG. 2. FIG. 2 is a flowchart illustrating an example of an overall processing flow of the information processing device 100 according to the present embodiment.

As illustrated in FIG. 2, in step S11, the user determines the fluorescence reagent 10A and the specimen 20A to be used for analysis. For example, the user prepares a pathology slide (a slice). In step S12, the user stains the specimen 20A using the fluorescence reagent 10A to prepare the fluorescence stained specimen 30A.

In step S13, the image acquisition unit 112 of the information processing device 100 images the fluorescence stained specimen 30A to acquire image information. In step S14, the analysis unit 131 separates an autofluorescence signal of the specimen 20A and a fluorescence signal of the fluorescence reagent 10A from the image information based on specimen information and reagent information and the image generation unit 132 generates image information using the separated fluorescence signal. For example, the image generation unit 132 generates image information in which the autofluorescence signal is removed from the image information or generates image information indicating the fluorescence signal for each of fluorescent dyes. Note that the information acquisition unit 111 acquires reagent information and specimen information from the database 200 based on the reagent identification information 11A added to the fluorescence reagent 10A used for generating the fluorescence stained specimen 30A and the specimen identification information 21A added to the specimen 20A.

In step S15, the space analysis unit 133 analyzes a biomarker from the image information after the color separation. For example, the space analysis unit 133 determines a phenotype of a cell. In step S16, the space analysis unit 133 performs dimension reduction (clustering) having positional information of multi-biomarkers. For example, the space analysis unit 133 performs the dimension reduction having the positional information of the multi-biomarkers and executes correlation analysis among biomarkers. In step S17, the space analysis unit 133 extracts a feature value from a correlation among the biomarkers. In step S18, the space analysis unit 133 executes effect prediction of a drug (medicine) using the feature value and patient information. For example, the space analysis unit 133 performs optimal drug selection, drug effect prediction, and the like using the feature value and the patient information. The patient information may include, for example, patient identification information and information such as administered medicine candidates for a patient. The processing in steps S15 to S18 and a configuration of the space analysis unit 133 concerning the processing are explained in detail below.

Note that the display processing unit 134 may generate, for example, image information including an analysis result (for example, a correlation analysis result) obtained in step S16, image information including the feature value obtained in step S17, and image information including an optimal drug selection result or an optimal drug effect prediction result obtained in step S18. These kinds of image information are displayed by the display unit 140. Accordingly, the user can view and grasp the various kinds of information displayed by the display unit 140.

The steps in the flowchart illustrated in FIG. 2 do not always need to be processed in time series in the described order. That is, the steps in the flowchart may be processed in order different from the described order or may be processed in parallel. The information processing device 100 may also execute processing not illustrated in FIG. 2.

### 1-3. Processing Example of Correlation Analysis of Multi-Biomarkers

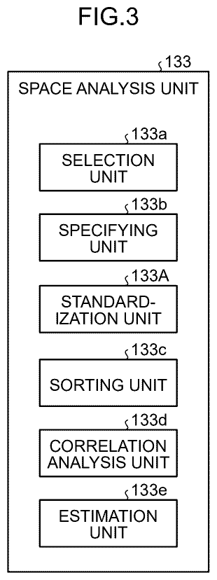

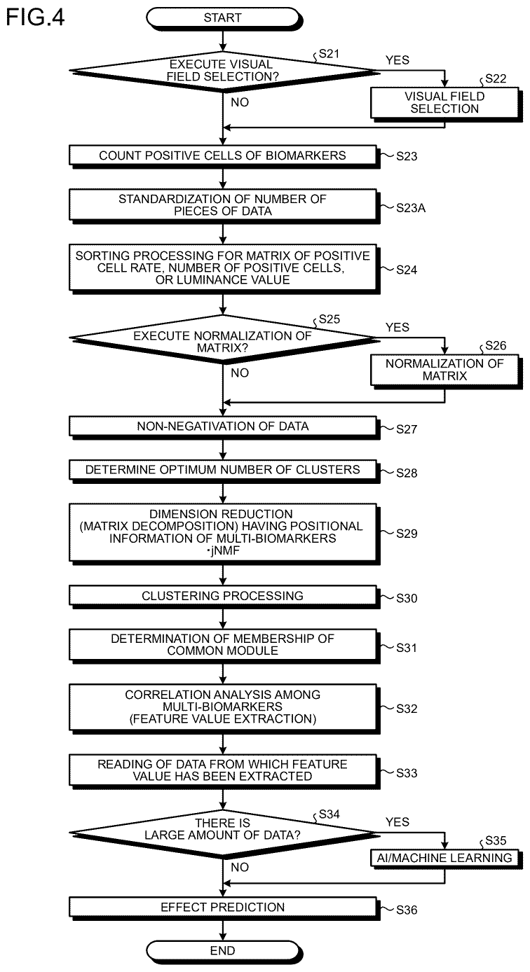

Processing examples of correlation analysis of multi-biomarkers according to the present embodiment are explained with reference to FIG. 3 and FIG. 4. FIG. 3 is a diagram illustrating an example of a schematic configuration of the space analysis unit 133 according to the present embodiment. FIG. 4 is a flowchart illustrating an example of a flow of correlation analysis processing of multi-biomarkers according to the present embodiment.

As illustrated in FIG. 3, the space analysis unit 133 includes a selection unit 133a, a specifying unit 133b, a standardization unit 133A, a sorting unit 133c, a correlation analysis unit 133d, and an estimation unit 133e.

The selection unit 133a determines a predetermined region (for example, a region of interest) of a sample (for example, a specimen image). The specifying unit 133b extracts, from a fluorescence spectrum deriving from a biological sample in a predetermined region (for example, a predetermined field of view), pieces of information (for example, an amount of positive cells) concerning a plurality of different biomarkers of a biological sample linked with positional information of the biological sample in the predetermined region and specifies the information. In the pieces of information concerning the biomarkers, the standardization unit 133A standardizes the number of pieces of data, which is the number of a plurality of pieces of unit information (for example, the number of blocks) included in the pieces of information concerning the biomarkers. The sorting unit 133c changes, based on the arrangement order of a plurality of pieces of unit information (for example, blocks) included in information concerning one biomarker among in the pieces of information concerning the plurality of biomarkers after the standardization, the arrangement order of a plurality of pieces of unit information (for example, blocks) included in information concerning the other biomarkers after the standardization. The correlation analysis unit 133d performs clustering processing on the pieces of information concerning the plurality of biomarkers in which the arrangement order of the unit information is changed and outputs a correlation among the pieces of information concerning the plurality of biomarkers. The estimation unit 133e estimates responsiveness to the patient of the administered medicine candidates from the correlation among the pieces of information concerning the plurality of biomarkers and the administered medicine candidates for the patient.

Here, the acquisition unit 110 (see FIG. 1) acquires the fluorescence spectrum deriving from the biological sample and positional information of the biological sample from a sample including the biological sample. The saving unit 120 saves the fluorescence spectrum deriving from the biological sample and the positional information of the biological sample. The fluorescence spectrum deriving from the biological sample and the positional information of the biological sample are used by the selection unit 133a. The acquisition unit 110 (see FIG. 1), that is, the information acquisition unit 111 acquires the administered medicine candidates for the patient concerning the biological sample. The saving unit 120 saves the administered medicine candidates for the patient concerning the biological sample. Information concerning the administered medicine candidates for the patient concerning the biological sample is used by the estimation unit 133e.

As illustrated in FIG. 4, in step S21, the selection unit 133a determines whether to execute visual field selection (determination of a predetermined region) on the specimen image after the color separation. In step S22, the selection unit 133a executes the visual field selection. In step S23, the specifying unit 133b counts the positive cells of the biomarkers in the specimen image after the color separation or the selected visual field of the specimen image. For example, the specifying unit 133b divides the specimen image after the color separation or the selected visual field of the specimen image into matrix-shaped blocks (block regions) and calculates a positive cell rate, the number of positive cells, or a luminance value for each of the blocks. Accordingly, a matrix of the positive cell rate, the number of positive cells, or the luminance value is calculated. Matrix information also includes positional information. Note that the positive cell rate is the number of positive cells with respect to the number of cells present per unit area. The number of positive cells is synonymous with the number of cells per unit area, that is, positive cell density. The positive cell rate, the number of positive cells, or the luminance value is information concerning a degree of positive cells (for example, information concerning an amount of positive cells) and is an example of information concerning biomarkers.

In step S23A, the standardization unit 133A standardizes the number of pieces of data in the matrix of the positive cell rate, the number of positive cells, or the luminance value. In step S24, the sorting unit 133c performs, based on a positive cell rate, the number of positive cells, or a luminance value of a certain biomarker, sorting processing on a matrix after the standardization of a positive cell rate, the number of positive cells, or a luminance value of the other biomarkers. In step S25, the correlation analysis unit 133d determines whether to execute normalization the matrix. In step S26, the correlation analysis unit 133d executes the normalization of the matrix. In step S27, the correlation analysis unit 133d makes data of the matrix non-negative. In step S28, the correlation analysis unit 133d determines an optimum number of clusters. For example, the optimum number of clusters may be automatically determined by the correlation analysis unit 133d or may be set in response to input operation of the user to the operation unit 160.

In step S29, the correlation analysis unit 133d executes matrix decomposition processing on the data of the matrix. For example, the correlation analysis unit 133d executes dimension reduction (simultaneous decomposition of a plurality of matrices) having positional information of multi-biomarkers with jNMF (joint Non-negative Matrix Factorization: JNMF). In step S30, the correlation analysis unit 133d executes clustering from a result of the dimension reduction. In step S31, the correlation analysis unit 133d determines membership of the common module. In step S32, the correlation analysis unit 133d performs correlation analysis among the multi-biomarkers. For example, the correlation analysis unit 133d extracts a feature value. In step S33, the estimation unit 133e reads data from which the feature value has been extracted. In step S34, the estimation unit 133e determines whether there is a large amount of data. In step S35, the estimation unit 133e executes AI/machine learning. In step S36, the estimation unit 133e executes effect prediction.

Here, in step S26, when the magnitude of a value is greatly different among samples or among biomarkers, the magnitude of matrices is normalized such that a sum of squares of the matrices is the same. In step S35, the estimation unit 133e can read the extracted feature value and determine a phenotype of cells. The estimation unit 133e assumes a phenotype of cancer of the patient together with the patient information and executes optimal drug (medicine) selection and drug effect prediction or uses the phenotype for patient selection in a clinical trial or the like. The estimation unit 133e functions as a predictor with AI/machine learning. Note that, when effect prediction is performed, prediction by AI or the like may be executed from the extracted feature value.

Note that the steps in the flowchart illustrated in FIG. 4 do not always need to be processed in time series in the described order. That is, the steps in the flowchart may be processed in order different from the described order or may be processed in parallel. The information processing device 100 may also execute processing not illustrated in FIG. 4.

### 1-4. Specific Example of Correlation Analysis of Multi-Biomarkers

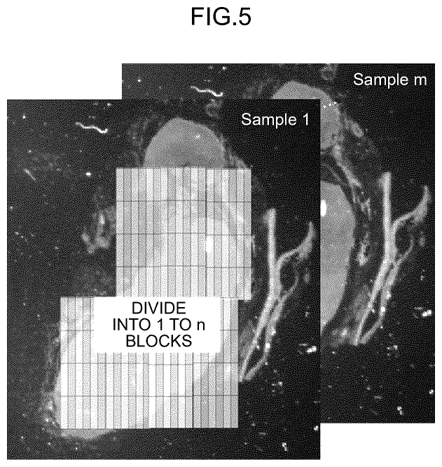

A specific example of correlation analysis of multi-biomarkers with respect to a specimen image according to the present embodiment is explained with reference to FIG. 5 to FIG. 10.

FIG. 5 is a diagram for explaining an example of a sample according to the present embodiment. As illustrated in FIG. 5, a plurality of specimens (for example, Samples 1 to m) are used. These specimens (specimen images) are, for example, samples of tonsils. Specifically, a plurality of tonsil samples stained with AF488_CD7, AF555_CD3, AF647_CD5, and DAPI (4′, 6-Diamidino-2 phenylindole, dihydrochloride) are used.

### Visual Field Selection Processing

The selection unit 133a sets a visual field for each of the specimens (for example, Samples 1 to m) and divides the visual field into one to n blocks. The visual field is a predetermined region (a region of interest). This predetermined region is set in advance by input operation of the user to the operation unit 160 but may be changeable by the input operation of the user to the operation unit 160. Note that positional information of regions in one specimen is two-dimensional information (positional information in a plane) and positional information of regions in a plurality of specimens is three-dimensional information (spatial information). For example, the positional information includes XY coordinates, Z coordinates, and the like based on pixels.

Here, the size of the field of view is sometimes different for each of specimens (for example, samples). When the size of the field of view is different for each of specimens, if all block sizes for each of the specimens are a predetermined size and are the same, the number of blocks is different for each of the specimens. When the number of blocks is different for each of the specimens, in order to carry out clustering processing such as jNMF, it is necessary to perform standardization processing of aligning the number of blocks for each of the specimens, that is, the number of pieces of data in the matrix. This standardization processing is explained in detail below. Note that the visual field, the number of blocks, the block size, and the like are set in advance by input operation of the user to the operation unit 160 but may be changeable by input operation of the user to the operation unit 160.

### Positive Cell Amount Calculation Processing

The specifying unit 133b calculates a positive cell rate of biomarkers for each of regions (blocks). For example, the specifying unit 133b calculates a positive cell rate (%) of the biomarkers for each of the regions. Accordingly, for example, individual positive cell rates of AF488_CD7, AF555_CD3, and AF647_CD5 are obtained. Note that the specifying unit 133b may calculate a numerical value other than the positive cell rate and may calculate, for example, an average luminance value, the number of positive cells, or the like in the region.

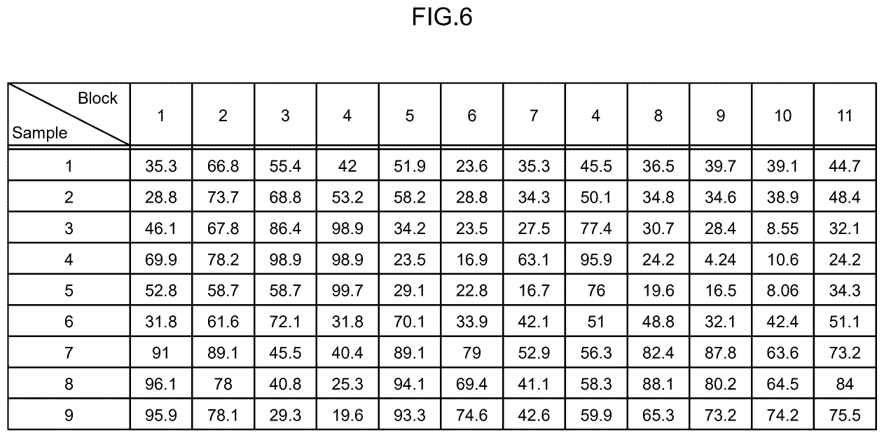

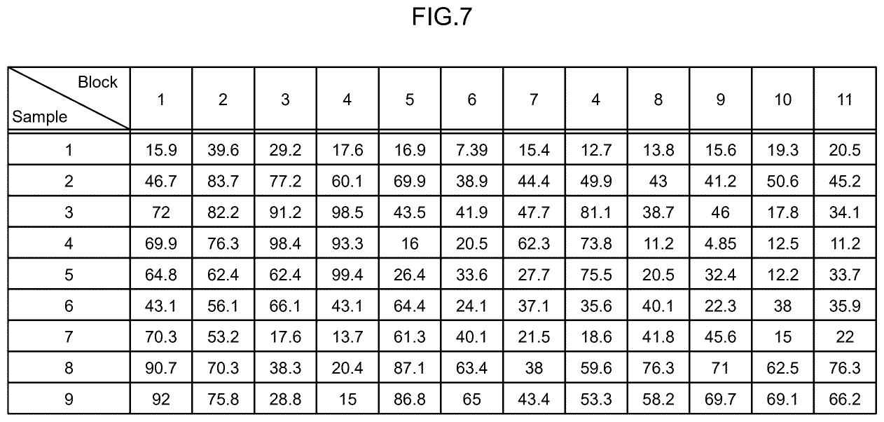

FIG. 6 is a diagram illustrating an example of a positive cell rate for each of blocks of AF488_CD7 according to the present embodiment. FIG. 7 is a diagram illustrating an example of a positive cell rate for each of blocks of AF555_CD3 according to the present embodiment. In the examples illustrated in FIG. 6 and FIG. 7, sample names and block names are indicated by “numbers”, the number of samples is nine, and the number of blocks is eleven. The number of samples and the number of blocks are only exemplifications and are not particularly limited. Here, the number of blocks for each of the samples is the same.

### Sorting Processing

The sorting unit 133c sorts, based on a positive cell rate of a specific biomarker, blocks (spaces) of other biomarkers for each of samples. For example, the sorting unit 133c sorts, based on the positive cell rate of the specific biomarker, the blocks of the other biomarkers in the row direction for each of the samples. Specifically, the sorting unit 133c rearranges the blocks of AF555_CD7 according to the arrangement order of the blocks in which positive cell rates of AF488_CD3 are in the descending order. The sorting unit 133c rearranges the blocks of AF555_CD5 according to the arrangement order of the blocks in which positive cell rates of AF647_CD3 are in the descending order.

At the time of the rearrangement explained above, the sorting unit 133c rearranges the blocks based on block names (for example, numbers 1, 2, 3, . . . ). After the rearrangement, the block names (the blocks) are arranged in the same order in AF555_CD3 and AF647_CD7. The same applies to AF555_CD3 and AF647_CD5 and block names (blocks) are arranged in the same order after rearrangement. Note that since a positive cell rate of AF555_CD3 serves as a sorting reference, AF647_CD5 functions as a reference marker. The reference marker is a reference marker for sorting. When the jNMF is carried out, by sorting a marker A (for example, AF488_CD7) and a marker B (for example, AF647_CD5) respectively with the reference marker, for example, the positions of the blocks can be made common even among a plurality of markers.

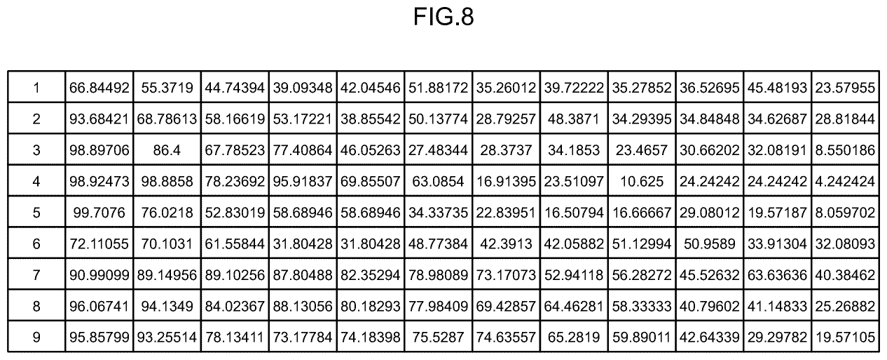

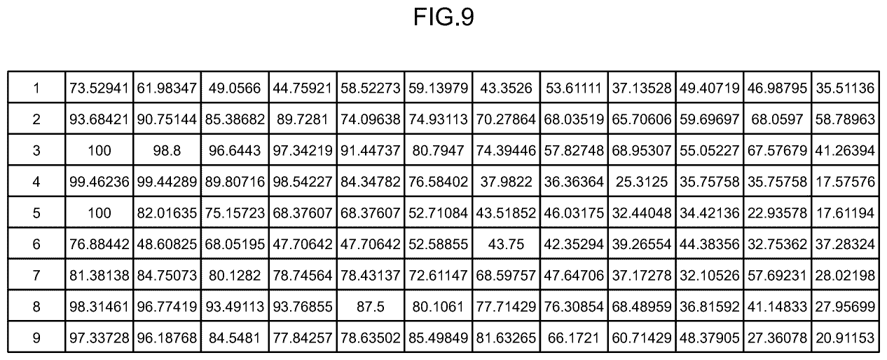

FIG. 8 is a diagram illustrating an example of a positive cell rate for each of blocks after sorting of AF488_CD7 according to the present embodiment. FIG. 9 is a diagram illustrating an example of a positive cell rate for each of blocks after sorting of AF647_CD5 according to the present embodiment. As illustrated in FIG. 8, the blocks of AF488_CD7 are arranged in the order of arrangement of blocks in which positive cell rates of AF555_CD3 is in the descending order. As illustrated in FIG. 9, the blocks of AF647_CD5 are also arranged in the order of arrangement of the blocks in which the positive cell rates of AF555_CD3 is in the descending order.

### Matrix Decomposition Processing Retaining Positional Information

The correlation analysis unit 133d carries out matrix decomposition processing on data of a sorted and rearranged matrix, for example, matrix decomposition processing corresponding to the combination of the plurality of biomarkers explained above. Here, since all values are positive cell rates, normalization of the matrix is not performed and, since all the values are positive values, the processing of making data of the matrix non-negative is also skipped. For example, the correlation analysis unit 133d processes two matrices with the jNMF and performs matrix decomposition (dimension reduction). At this time, the correlation analysis unit 133d performs simultaneous decomposition of a plurality of matrices while retaining positional information (spatial information). Note that the correlation analysis unit 133d acquires pieces of information concerning biomarkers and information such as the number of clusters k as input data.

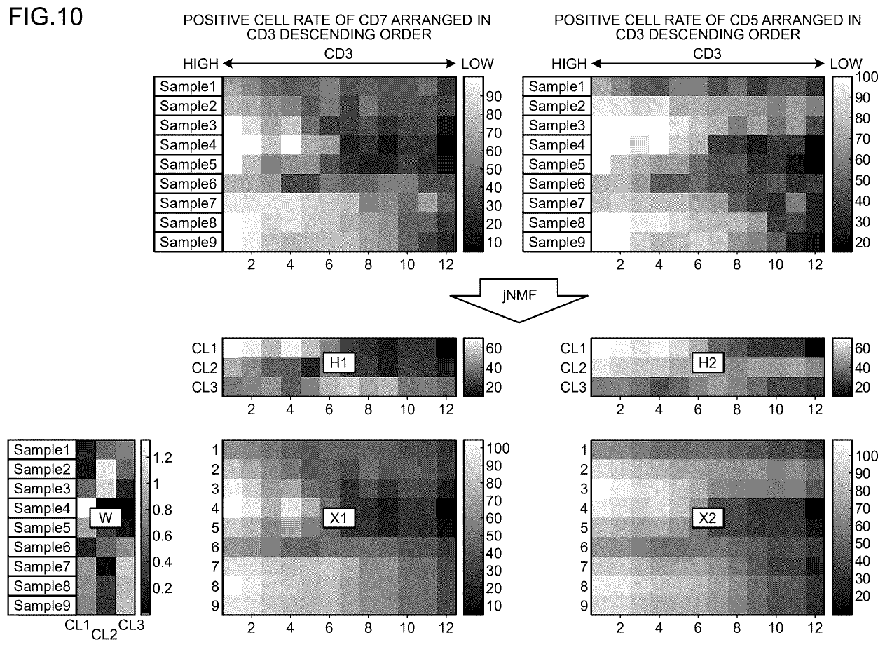

FIG. 10 is a diagram for explaining an example of the jNMF according to the present embodiment. In the example illustrated in FIG. 10, the number of clusters k is set to, for example, k=3. Note that the number of clusters k is set as appropriate but may be calculated by an elbow method or the like. In the example illustrated in FIG. 10, CD3 is AF555_CD3, CD5 is AF647_CD5, and CD7 is AF488_CD7. Hereinafter, AF555_CD3 is sometimes referred to as CD3, AF647_CD5 is sometimes referred to as CD5, and AF488_CD7 is sometimes referred to as CD7.

Here, the jNMF (joint NMF) is an extension of NMF (Non-negative Matrix Factorization). This jNMF can target a plurality of matrices and enables integrated analysis of multi-omics data. The NMF on which the jNMF is based is decomposing one matrix into two small matrices. Here, a certain matrix is an N×M matrix X and the matrix X can be represented as a product of matrices W and H. Specifically, the NMF decomposes a non-negative matrix X of N rows and M columns (N×M) into a non-negative matrix W of N rows and k columns (N×k) and a non-negative matrix H of k rows and M columns (k×M) (X=WH). For example, the matrix W and the matrix H are determined such that a root mean square residual D between the matrix X and a product (W*H) of the matrix W and the matrix H is minimized. k is the number of clusters. Note that the NMF is a method that can emphasize a relevance between matrix elements by decomposition of potential elements rather than explicit clustering and is suitable further for capturing outliers such as mutation and overexpression.

As a method of the matrix decomposition processing, besides the jNMF explained above, INMF (Infinite NMF), MCCA (Multiple Canonical Correlation Analysis), MB-PLS (Multi-Block Partial Least-Squares), JIVE (Joint and Individual Variation Explained), and the like can be used.

Here, in the example illustrated in FIG. 10, three classes (clusters) are CL1, CL2, and CL3. CL1 is a first column of W and a first row of H1 and H2. CL2 is a second column of W and a second row of H1 and H2. CL3 is a third column of W and a third row of H1 and H2. Here, data is divided into a common basis vector W and feature vectors H1 and H2. Note that blocks (block regions) and pieces of data of a matrix correspond to each other. The matrix W is also referred to as common basis matrix. When the jNMF is carried out, a plurality of matrices can be decomposed into the common basis matrix W and feature vectors H of markers. Columns of the matrix W represent clusters and samples with high values are classified into clusters.

### Clustering Processing

The correlation analysis unit 133d classifies samples into clusters based on a value of the common basis vector W and determines membership (clustering). In determining the membership for each of the clusters, a region where a value is equal to or larger than a threshold may be determined as the membership of the cluster or the membership of the cluster may be determined from a Z score.

### Extraction of a Common Module

The correlation analysis unit 133d extracts, for each of the clusters, as membership of a common module, a region (a block) where a value of a feature vector is high. For example, the correlation analysis unit 133d extracts a feature value (for example, a positive rate) of a cell for each of common modules based on the correlation of the biomarkers, that is, the membership of the common module for each of the cluster. The common module is a region extracted as membership relating to a clustering result. The membership is a constituent element extracted as a common feature value relating to the clustering result and is, for example, a constituent region (for example, a region or a block) extracted as the common feature value.

In determining the membership of the common module, a region in which a value is equal to or larger than the threshold may be determined as the membership of the common module or the membership of the common module may be determined from a Z score. The common module is a region extracted as a region having a characteristic in common among samples in a result of the jNMF. This region has a high degree of contribution to the cluster.

In the example illustrated in FIG. 10, in CL1, samples 3 to 5 are a main region and includes Samples 7 to 9 as well, but, as membership of a common module of CL1, a region where CD3 is high and CD7 is also high and CD3 is high and CD5 is high in the region of the samples 3 to 5 is extracted. In CL2, samples 1 to 3 are classified and, as membership of a common module, a region where CD3 is high and CD7 is also high and CD3 is high and CD5 is high in the region of the samples 1 to 3 is extracted. In CL3, a region of the Samples 7 to 9 is classified. A feature value (for example, a positive rate) of a cell for each of common modules is extracted based on the classification of the samples for each of the clusters explained above.

As in the result explained above, the clusters can be divided for each of the samples according to a slight difference of a positive cell rate. The region where CD3 is high and CD7 is also high and CD3 is high and CD5 is high can be extracted as having a correlation. Note that, since CD3, CD5, and CD7 are markers of a T cell, the same result as an expected result was successfully obtained.

According to a series of correlation analyses multi-biomarkers explained above, it is possible to analyze an interaction between positional information and positive cells of the multi-biomarkers by performing the clustering processing on a specimen image stained with a plurality of biomarkers while retaining the positional information. That is, it is possible to analyze and acquire a correlation between different biomarkers from a positive rate and positional information of the different biomarkers.

Note that, in the above explanation, one visual field is designated for each of the specimens. However, not only this, but, by designating a plurality of visual fields for each of the specimens, it is possible to extract a feature value of a cell for each of the visual fields even in the same specimen. It is also possible to perform comparison for each of the specimens using a whole slide of the specimen and apply a result of the comparison to patient selection. By using different specimens as samples (for example, tonsils, lymph, a large intestine, a bone marrow, and skin), it is possible to examine a correlation between a common feature value and multi-markers of different cancer cells and apply a result of the examination to, for example, medicine prediction for a type of cancer.

### Determination of the Number of Clusters

The correlation analysis unit 133d can determine, for example, the number of clusters k from an error tendency of a residual. The correlation analysis unit 133d is capable of calculating a residual: sum of squares (SSE) of the jNMF while changing the number of clusters k and calculating an optimum number of clusters k from a change tendency of the residual sum of squares. Note that, when it is hard to see the change tendency when calculating the optimum number of clusters k, the optimum number of clusters k can be further calculated by a method such as an elbow method. The elbow method is a method of finding a combination in which both of an SSE and the number of clusters k are as small as possible. Note that, for example, the number of clusters k that minimizes the residual and the Euclidean distance may be set or the number of clusters k may be set as the number of clusters that the user desires to se. That is, the number of clusters k may be able to be set by input operation of the user to the operation unit 160.

### Determination of Membership of a Cluster and Membership of a Common Module

When desiring to always cause a sample to belong to one cluster for each of samples or spaces, the correlation analysis unit 133d can set the cluster from a maximum value. However, depending on a sample, it is conceivable that the sample belongs to a plurality of clusters or does not belong to all the clusters. Therefore, it is possible to obtain membership of the cluster from a Z score.

For example, the correlation analysis unit 133d calculates Z scores (Zᵢⱼ) of elements of columns of W and rows of H using a relational expression Zᵢⱼ=(Xᵢⱼ−Uᵢ)/σᵢ. Here, Uᵢ is an average or a median value of positive cell rates, the numbers of positive cells, luminance values, or the like of biomarkers in Hi (i=1, 2, 3, . . . ). σᵢ is a standard deviation or a median absolute deviation. If Zᵢⱼ is larger than a threshold T, the correlation analysis unit 133d allocates Zᵢⱼ as membership of a common module. The threshold T is set in advance. The threshold T may be set to two or more values from statistical superiority or may be set to a value more suitable for the user from a tendency of the membership of the cluster. The threshold T may be capable of being set by input operation of the user to the operation unit 160. Note that, as an expression for feature value extraction other than the Z score, it is also possible to use a feature value=Xᵢⱼ−Uᵢ (a difference from an average) or a feature value=Xᵢⱼ−Uᵢ/Uᵢ (not divided by the standard deviation but divided by an average value).

### Confirmation of a Correlation of Membership Allocation of a Cluster

In order to evaluate the stability of clustering, the correlation analysis unit 133d may perform correlation analysis using a Pearson's correlation coefficient, pairwise correlation analysis, or the like in order to check whether features of processing results of respective types of clustering processing are correlated.

### Cloud Cooperation

Various types of processing (for example, visual field selection processing, positive cell amount calculation processing, sorting processing, and clustering processing) such as correlation analysis processing for multi-biomarkers and prediction processing for medicine effects can be executed by software on a cloud side by carrying out the various types of processing on the cloud side in cooperation with various kinds of data.

Note that, in the embodiment explained above, three types of biomarkers are used. However, not only this, but, for example, two or more types of biomarkers may be used. A biomarker used for sorting may be, for example, an immune cell marker or may be a tumor marker. Examples of the biomarker include a molecular biomarker and a cell biomarker.

### 1-5. Method for Standardizing the Number of Pieces of Data

### 1-5-1. Processing Example of Standardization of the Number of Pieces of Data

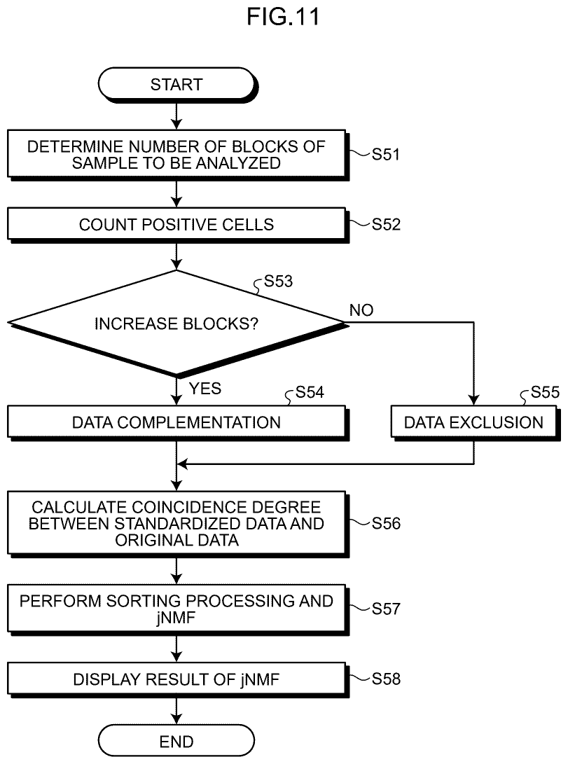

A processing example of standardization f the number of pieces of data according to the present embodiment is explained with reference to FIG. 11. FIG. 11 is a flowchart illustrating an example of a flow of standardization processing for the number of pieces of data according to the present embodiment.

The size of a tissue of a pathological specimen is sometimes different for each of samples. When the sizes of an imaging region of the pathological specimen are compared, for example, there may be a two to three fold difference between a minimum size and a maximum size. For this reason, as a method of complementing data, a complementation method that does not affect a result of the jNMF even when data two to three times as large as actual data is complemented is desirable. Here, in standardization of data, there are mainly two cases of a case in which the data is increased and a case in which the data is reduced. The sizes of samples desired to be analyzed with the jNMF are compared to determine the number of blocks (the number of pieces of data) between the samples.

As illustrated in FIG. 11, in step S51, the selection unit 133a executes visual field selection and determines the number of blocks of a sample to be analyzed. In step S52, the specifying unit 133b counts positive cells for each of the blocks. In step S53, the standardization unit 133A determines whether to increase the blocks. In step S54, the standardization unit 133A complements data corresponding to the blocks. In step S55, the standardization unit 133A excludes the data corresponding to the blocks. In step S56, the standardization unit 133A calculates a coincidence degree between the standardized data and the original data. In step S57, the sorting unit 133c performs sorting processing and the correlation analysis unit 133d carries out the jNMF. In step S58, the display unit 140 displays the result of the jNMF.

For example, the same number of pieces of data (number of blocks) is required in all specimens in carrying out the jNMF. The number of pieces of data is different depending on the size of a tissue of a pathological specimen. However, if the number of pieces of data is made equal to a minimum number of samples, when there is only a few pathologically characteristic regions, it is likely that the regions are lost. For this reason, it is desirable to simulatively generate data equivalent to the number of lacking data or to exclude data by the number of excess data such that there is no large difference between a population of the data before standardization and a population of the data after standardization, that is, there is no influence on a clustering result.

As explained above, with the technique of reducing the number of pieces of data according to the smallest sample, when there is only a few characteristic regions (cancer regions) with respect to the entire region, the characteristic regions are sometimes lost. For this reason, it is desirable to use all tissue regions for analysis without excluding regions other than a background region without tissues and a region where autofluorescence is too high. For example, in some case, data is complemented in order to equalize the number of pieces of data between samples. However, even when the data is complemented, it is desirable that the complementation does not affect the analysis result of the jNMF. In the display of the analysis result, when data of a certain part is complementary data when the result of the jNMF is displayed, the data sometimes cannot be associated with an actual pathological image, that is, there is sometimes no actual data that can be displayed. For example, the complemented data is data not associated with a pathological image of actual data, and a pathological image corresponding to the complemented data cannot be presented to the user.

As a method of equalizing the number of pieces of data between samples, for example, there are a method of generating data having features similar to features of an original population and complementing data (complementation of information) and a method of excluding a region not relating to analysis (exclusion of information). When data is complemented, the data is complemented using, for example, the data is complemented using an average value and a standard deviation of the original population such that features of a population of the data before the complementation and a population of the data after the complementation do not greatly change (for example, a difference between the population of the data before the complementation and the population of the data after the complementation does not exceed a predetermined threshold). When the number of pieces of data is reduced, data (a block) of a background region without tissues in the pathological image is excluded or data (a block) of a region where a luminance value is equal to or larger than a predetermined threshold (a region where autofluorescence is high) is excluded.

According to the complementation (data complementation) or the exclusion (data exclusion) of the information explained above, analysis can be executed by analysis processing such as the jNMF even between specimens having different tissue sizes. By carrying out the jNMF, clustering can be performed for each of features of the spatial region of the sample. For example, it is possible to carry out the jNMF without losing pathological feature regions. The difference between the population and the complemented data is small and the influence on the clustering result is smaller than the influence in the normal missing value data complementation method. On a result display screen, the user can visualize the complementary data and real data and can refer to real data closest to the complementary data. For example, it is possible to improve the visibility of the user by presenting a pathological image corresponding to the real data closest to the complementary data or presenting the real data and the complementary data in a distinguishable manner. The data complementation method, the data exclusion method, the display method, and the like explained above are explained in detail below.

### 1-5-2. Data Complementation Method

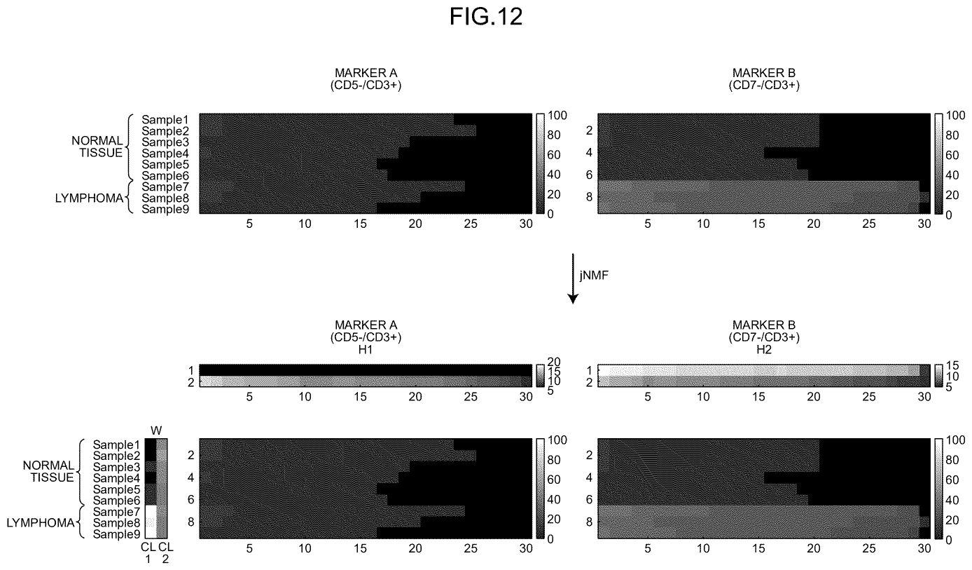

A data complementation method according to the present embodiment is explained with reference to FIG. 12 to FIG. 18.

### jNMF Simulation of a Pathological Specimen

Simulation data simulating a T-cell lymphoma (CD7 deletion) was created. Due to characteristics of the jNMF, a feature value having a larger (increasing) value has higher cluster detection sensitivity than a feature value having a lower (decreasing) value. Therefore, it is assumed that a missing rate of a lymphoma is observed this time. As markers (T cell markers) to be used, a marker A and a marker B are used. As the marker A, the number of CD5− cells/the number of CD3+ cells is used. As the marker B, the number of CD7− cells/the number of CD3+ cells is used. As a reference marker for sorting, the number of CD3+ cells is used. The current simulation data is sorted in marker descending order of the simulation data itself. The number of blocks is thirty.

Note that, in order not to complicate a simulation, the number of CD5− cells/the number of CD3+ cells and the number of CD7− cells/the number of CD3+ cells are selected as the markers. However, in order to more accurately count lymphocytes (T cells), the number of CD3+CD5− cells/the number of CD3+ cells may be used as the marker A and the number of CD3+CD7− cells/the number of CD3+ cells may be used as the marker B. Since there are autofluorescence and leakage from other channels when an actual specimen is used, it can be said that examining a cell ratio of CD7− or CD5− in CD3+ cells more reflects the number of target T cells. However, this does not apply to a CD3− specimen.

### Data Set 1

Twelve samples were prepared. Six samples (Sample 1 to 6) are normal tissues. In the Samples 1 to 6, a missing rate was set at μ=7.5% and σ=2.5% such that CD5+ and CD7+ were respectively between 100% and 85% with respect to CD 3+. Three samples (Samples 7 to 9) are T-cell lymphomas (CD7 deletion). In the Samples 7 to 9, the missing rate was set at μ=7.5% and σ=2.5% such that CD5+ was respectively between 100% and 85% with respect to CD3+. This is the same as in the normal tissues. A CD7 missing rate in a block was set at μ=30%, σ=3% and a lymphoma region of the entire samples was set at 96.7%. Three samples (Samples 10 to 12) are T-cell lymphomas (CD7 deletion) that require data complementation. As the Samples 10 to 12, samples having only fifteen blocks, which is half the number of small blocks as compared with the Samples 1 to 9, were prepared. Missing rates of the lymphomas and the normal tissues are the same as the missing rate of the Samples 7 to 9. Lacking data equivalent to fifteen blocks are increased to data equivalent to thirty blocks by data complementation and analysis is performed.

It is seen, from the studies of characteristic tests of the jNMF using various simulation data up to now, that it is possible to cluster even a combination of an extreme data set with the lymphomas and the normal tissues if the missing rate in the block is 30%. Thus, in the current simulation as well, as the strictest condition, the CD7 missing rate in the block was set as 30% and the lymphoma region in the entire samples was set as 96.7%.

FIG. 12 is a diagram for explaining an example of jNMF results in a data set (Samples 1 to 9) without complementation according to the present embodiment. In the example illustrated in FIG. 12, results of the jNMF of the Samples 1 to 9 not subjected to data complementation are illustrated. As illustrated in FIG. 12, looking at W, which is a common basis matrix, in CL1, lymphomas of the Samples 7 to 9 are clustered and clusters are separated in the normal tissues and the lymphomas. From the results explained above, it is seen that, in order to perform data complementation not to affect a clustering result of the jNMF, a data complementation method is desirable in which lymphomas (Samples 7 to 9) and data-complemented lymphomas (Samples 10 to 12) are classified into the same cluster and classified into a cluster different from a cluster of normal tissues (Samples 1 to 6).

### Data Complementation Method According to a Comparative Example

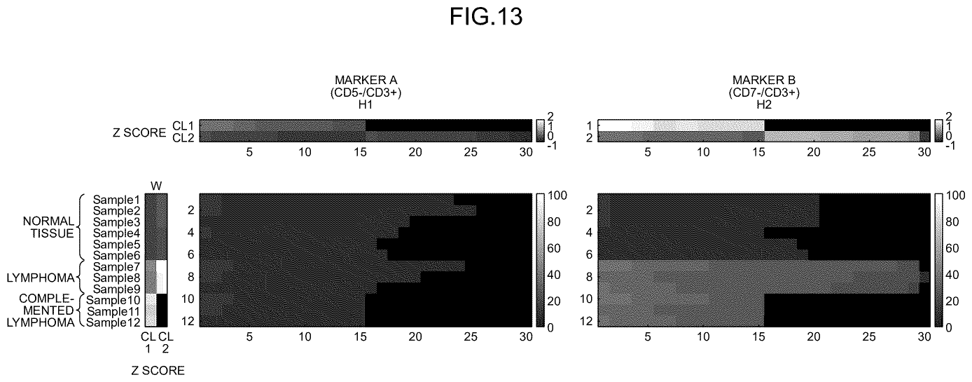

FIG. 13 is a diagram for explaining an example of jNMF results in a data set 1 complemented by a data complementation method according to a comparative example. In the data complementation method according to the comparative example, the number of lacking data equivalent to the fifteen blocks of the Samples 10 to 12 were complemented with 0. As illustrated in FIG. 13, looking at W, which is a common basis matrix, it is seen that clusters are separated in the lymphomas (the Samples 7 to 9) and the complemented lymphomas (the Samples 10 to 12) and it is not desirable to complement data with 0. However, the complementing data with 0 is not completely denied and, depending on various conditions, it is effective to complement some data (for example, one or several pieces of data) with 0.

### Data Complementation Method According to the Present Embodiment

From the results of the studies up to the present, it is seen that, in the jNMF, data with a small standard deviation is likely to be subjected to cluster extraction when an average of a data set is the same value and data with a high average value is likely to be subjected to cluster extraction when a standard deviation is the same value. Since an average value and a standard deviation of data are important for cluster classification of the jNMF, a data complementation method in which the average value and the standard deviation of the data do not greatly change is used. For example, a method of complementing data with an average value or a mode value is conceivable besides the method of complementing lacking data with 0. However, as the number of pieces of data that has to be complemented increases, the data further deviates from an average value and a standard deviation of an entire original data group. Therefore, the method is assumed to be inappropriate when data has to be complemented two to three times or more as in the current case.

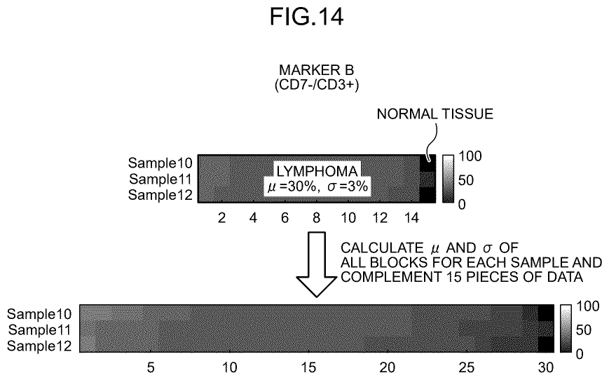

FIG. 14 is a diagram for explaining an example of the data complementation processing according to the present embodiment. The standardization unit 133A calculates an average value μ and a standard deviation σ of samples for each of the samples and complements data equivalent to fifteen blocks with random numbers having a normal distribution satisfying the calculated average value μ and calculated standard deviation σ. For example, as illustrated in FIG. 14, in each of the Samples 10 to 12, the standardization unit 133A calculates the average value μ and the standard deviation σ of data equivalent to all blocks (fifteen blocks) obtained by combining lymphoma regions and normal tissue regions of the sample, generates random numbers having a normal distribution satisfying the average value μ and the standard deviation σ of the data equivalent to the fifteen blocks, and increases the data equivalent to the fifteen blocks to data equivalent to thirty blocks in total. Since it is assumed that the number of CD3-positive cells is the reference marker this time, the complementary data and the original data were sorted together in descending order. The processing explained above is performed with the respective markers (for example, the two markers A and B).

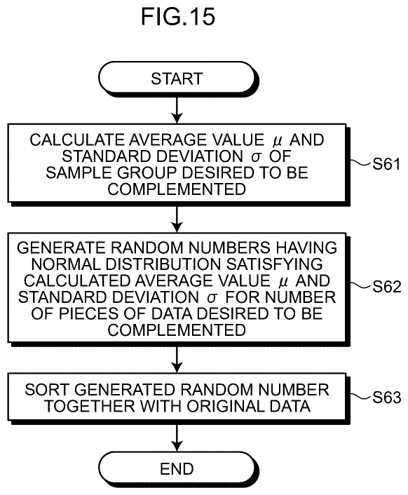

FIG. 15 is a flowchart illustrating an example of a flow of the data complementation processing according to the present embodiment. As illustrated in FIG. 15, in step S61, the standardization unit 133A calculates the average value μ and the standard deviation σ of a sample group desired to be complemented. In step S62, the standardization unit 133A generates random numbers having a normal distribution that satisfies the calculated average value μ and the calculated standard deviation σ by the number of pieces of data desired to be complemented. In step S63, the standardization unit 133A sorts the generated random number together with the original data according to the reference marker. Note that the difference between a population of samples desired to be complemented and complemented data is small and the influence on a clustering result is less significant than the influence in a normal missing value data complementation method.

### jNMF Results in the Data Set 1 Complemented by the Data Complementation Method According to the Present Embodiment

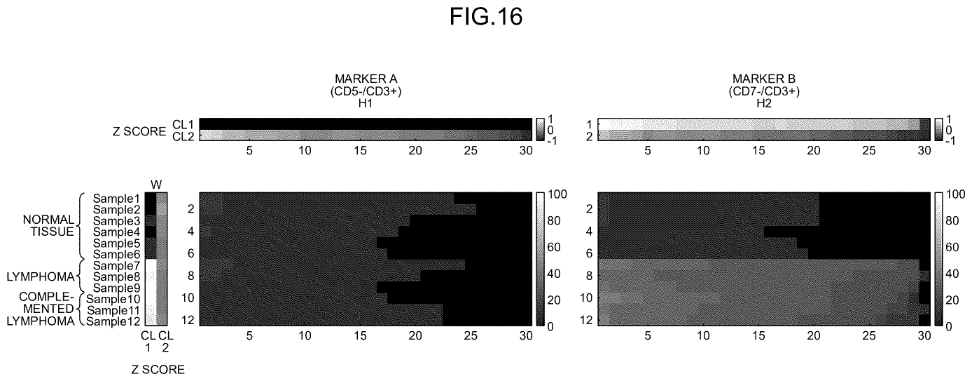

FIG. 16 is a diagram for explaining an example of jNMF results in the data set 1 complemented by the data complementation method according to the present embodiment. As illustrated in FIG. 16, lymphomas (Samples 6 to 9) and complemented lymphomas (Samples 10 to 12) were classified into the same cluster. This result is the same as a result at the time when the jNMF is carried out in the Samples 1 to 9 illustrated in FIG. 12. Therefore, it is seen that the data complementation method of the present disclosure is superior to the data complementation method of complementing data with 0.

In the simulation explained above, the lymphoma is assumed to be the sample in which the missing rate in the block is low but the lymphoma region spreads over the entire region. As a more difficult situation, assuming a sample in which a missing rate in a block is low and a lymphoma region is present only a little (data of a lymphoma closest to a normal tissue), it is checked below whether clustering can be performed between the normal tissue and the lymphoma.

### Data Set 2

Twelve samples were prepared. Six samples (Samples 1 to 6) are the same as those in the data set 1 explained above. Three samples (Samples 7 to 9) are the same as those in the data set 1 explained above. However, in the Samples 7 to 9, a CD7 missing rate in a block was set as μ=30%, σ=3% and a lymphoma region of the entire samples was set as 3.3%. Three samples (Samples 10 to 12) are the same as those in the data set 1 explained above. As in the data set 1 explained above, lacking data equivalent to fifteen blocks are increased to data equivalent to thirty blocks by data complementation and analysis is performed. Accordingly, jNMF results, which are analysis results, are obtained.

### jNMF Results in the Data Set 2 Complemented by the Data Complementation Method According to the Present Embodiment

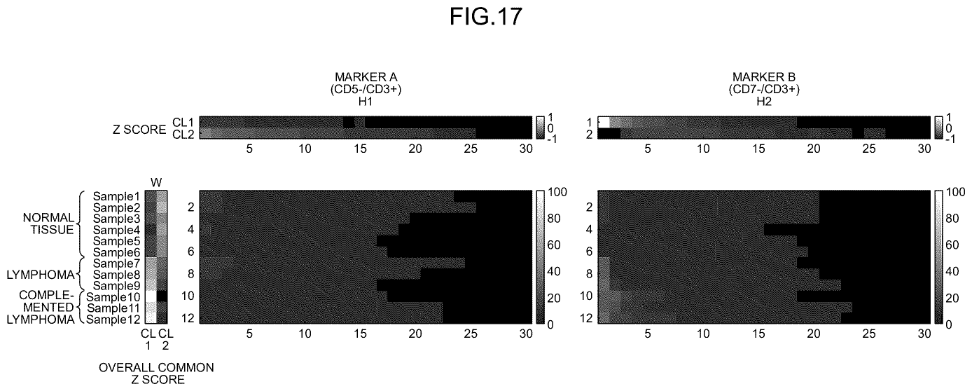

FIG. 17 is a diagram for explaining an example of jNMF results in the data set 2 complemented by the data complementation method according to the present embodiment. As illustrated in FIG. 17, looking at W, which is a common basis matrix, lymphomas (Samples 6 to 9) and complemented lymphomas (Samples 10 to 12) were classified into the same cluster and clustered from a normal tissue. Therefore, it is seen that the data complementation method according to the present embodiment has less significant influence on a clustering result than the normal data complementation method even in data complementation in a sample having only a slight difference between a normal tissue and a lymphoma and clustering can be correctly performed.

### Sorting Processing for Generated Data

There is processing of sorting data generated by complementing data by the number of lacking data together with actual data. Whether to sort the data in descending order or in ascending order is determined according to a combination of markers to be used and presence or absence of a correlation with a reference marker. A marker to be used is different depending on a use. The marker is selected according to a use such as lymphoma diagnosis, clustering by the cancer microenvironment, and patient selection of cancer immunotherapy.

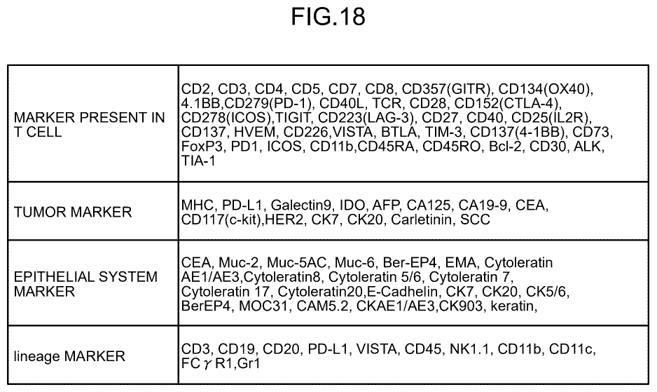

FIG. 18 is a diagram illustrating a plurality of types of markers according to the present embodiment. As illustrated in FIG. 18, there are a plurality of types of markers. These markers are only examples and the marker to be used is not limited to the markers illustrated in FIG. 18. The user can optionally select a combination of the markers. Note that, although FIG. 18 does not illustrate a cell proliferation marker Ki67 often used in benign/malignant diagnosis of cancer, the cell proliferation marker Ki67 may be used.

### Example 1 of a T-Cell Lymphoma

The marker A is set to the number of CD5+ cells, the marker B is set to the number of CD7+ cells, and the reference marker is set to the number of CD3+ cells. When the reference marker is set to the number of CD3+ cells (for example, the number of positive cells) and sorting is performed in the CD3 descending order, since CD5 and CD7 have a correlation with CD3, complemented data is also rearranged in the descending order. Since there are variations in actual data, the complemented data may be rearranged at a position closest to the actual data. For example, when a marker that is known to stain not T cells but B cells is used as a marker C (the number of CD20+ cells/the number of CD3+ cells), the complemented data is rearranged in the ascending order because CD20 is rearranged in the ascending order. Thus, whether the complemented data is rearranged in the descending order or the ascending order varies depending on a relationship between the reference marker and the markers.

The marker A may be set to the number of CD3+CD5+ cells and the marker B may be set to the number of CD3+CD7+ cells. As the markers, it is possible to use not only single positive but also double positive and triple positive numbers and ratios of positive cells. It is also possible to use not only the number of positive cells but also the number and a ratio of negative cells.

### Example 2 of the T-Cell Lymphoma

The jNMF can also be carried out at the missing rate the marker explained above. The marker A is set to the number of CD5− cells/the number of CD3+ cells, the marker B is set to the number of CD7− cells/the number of CD3+ cells, and the reference marker is set to the number of CD3+ cells. Since the number of CD3+ cells is the reference marker, the missing rate is different depending on a ratio of a lymphoma region of a sample. In this case, by not only the descending order sorting, complemented data may be rearranged at a position closest to actual data. For example, it is possible to automatically and optimally sort the complemented data again by reading a barcode of a marker set.

### Example 3 of the T-Cell Lymphoma

Since the detection sensitivity is different depending on a marker corresponding to a panel of a T-cell lymphoma or there is a difference in an amount of autofluorescence caused by leakage depending on a dye, there is an optimal combination of a dye and a marker. A combination of DAPI, AF488_CD7, AF555_CD3, eF615_CD20, AF647_CD5, and the like is preferable. For example, by reading a barcode corresponding to the panel, it is possible to automatically select the reference marker, the marker A, and the marker B and automatically and optimally sort the complemented data again.

### Example of a Cancer Microenvironment

The marker A is set to the number of CD3+ cells, the marker B is set to the number of PDL1+ cells, and the reference marker is set to the number of CK+ cells. The combination explained above is an exemplification and other combinations can be used.

### 1-5-3. Data Exclusion Method

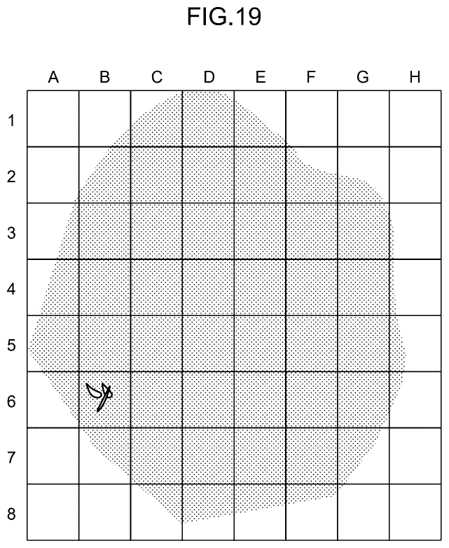

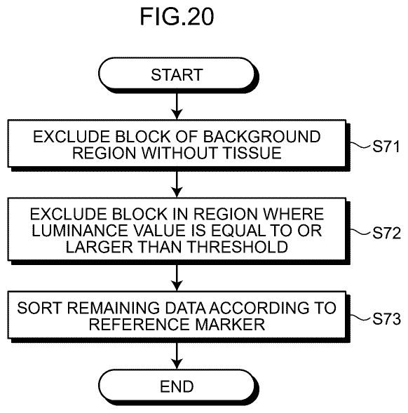

A data exclusion method according to the present embodiment is explained with reference to FIG. 19 and FIG. 20.

For example, when the number of blocks of a sample is larger than a set number of blocks, a region not related to analysis is excluded. For example, the standardization unit 133A excludes a block of a background region where no tissue is reflected in an imaged region (information concerning a region without a tissue). As determination of presence or absence of a tissue, there are (1) a method of excluding, from an analysis target, a region where a background region where a tissue is not reflected (a region where a luminance value is 0 in all dyes) is present by a predetermined threshold (for example, several %) or more and (2) a method of performing DAPI determination and excluding, from an analysis target, a region where the number of DAPIs is a predetermined threshold or less (for example, a region where no DAPI is present). These methods may be automated. The predetermined thresholds are set in advance by input operation of the user to the operation unit 160 but may be changeable by input operation of the user to the operation unit 160. The same applies to other thresholds.

FIG. 19 is a diagram for explaining an example of blocks of a sample according to the present embodiment. As illustrated in FIG. 19, for example, when it is determined that a block A1, a block G1, and a block H1 are regions having no tissue at first sight but an end of a tissue is slightly included as in a block F1, the user may set a predetermined threshold to set whether the block F1 is to be analyzed or excluded.

As a candidate of a block to be excluded from an analysis target, a block that is a region having an excessively high luminance value because of autofluorescence and not suitable for analysis, for example, a block B6 may be excluded. It is also possible that the user sets a threshold of a luminance value to automatically exclude a block having a luminance value is equal to or larger than the predetermined threshold and excessively high autofluorescence (a region having a luminance value equal to or larger than the predetermined threshold). As a block that is a region having no influence even if being excluded from the analysis target, for example, there is also a region defocused because of a deficiency of imaging.

FIG. 20 is a flowchart illustrating an example of a flow of data removal processing according to the present embodiment. As illustrated in FIG. 20, in step S71, the standardization unit 133A excludes a block of a background region where no tissue is imaged. In step S72, the standardization unit 133A excludes a block of a region where a luminance value is equal to or larger than the threshold. In step S73, the standardization unit 133A sorts the remaining data according to the reference marker. Accordingly, it is possible to automatically exclude blocks with the threshold set by the user and reduce the number of pieces of data to a target number.

### 1-6. Coincidence Degree Calculation Method

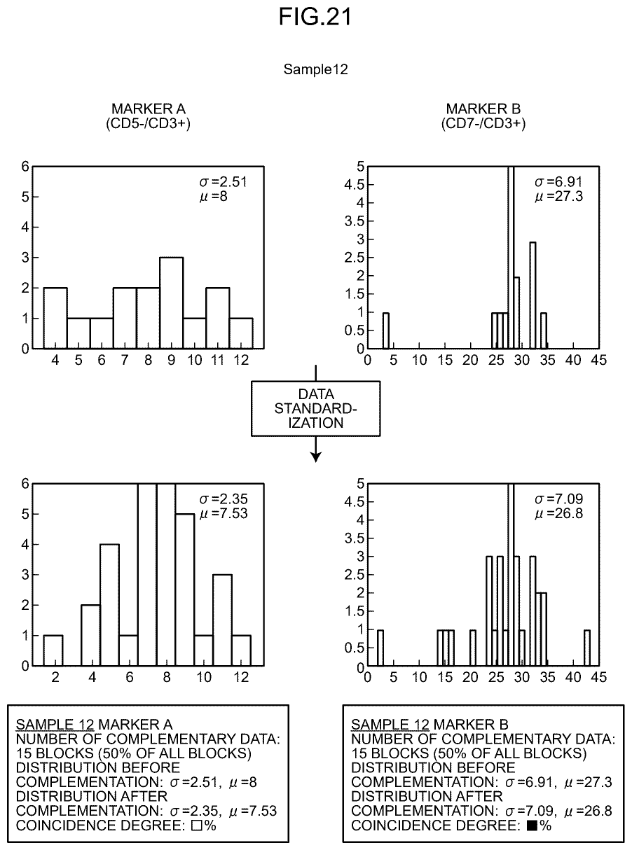

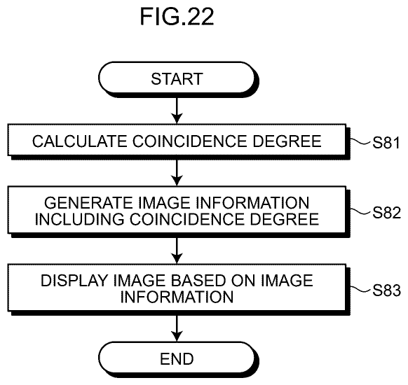

A coincidence degree calculation method according to the present embodiment is explained with reference to FIG. 21 and FIG. 22.

In the coincidence degree calculation method, a coincidence degree between original data and standardized data (data after standardization) is calculated with respective markers for each of samples. As this calculation method, for example, a chi-square test, a KS test, a t-test, a Mann-Whitney U test, a Euclidean distance, a Pearson's correlation coefficient, a crossing method, a KL divergence, or a Bhattacharyya distance is used. Visibility may be improved by presenting a histogram of data to the user. In addition, information indicating what percentage of an entire block has been subjected to data complementation or data has been excluded, the average value μ and the standard deviation σ before and after data standardization, or the like may be presented to the user. Here, an example of presenting a Sample 12 subjected to data complementation to the user is illustrated in FIG. 21.

FIG. 21 is a diagram for explaining an example of presenting a Sample 12 subjected to data complementation to the user according to the present embodiment. As illustrated in FIG. 21, information such as the average value μ and the standard deviation σ and the coincidence degree before and after data standardization is displayed by the display unit 140 for each of the marker A and the marker B. In the example illustrated in FIG. 21, the information such as the average value μ and the standard deviation σ and the coincidence degree before and after data standardization is displayed. However, only one of these kinds of information may be displayed. Note that a display image including the information such as the average value μ and the standard deviation σ and the coincidence degree before and after data standardization is generated by the display processing unit 134. The user grasps information such as the average value μ and the standard deviation σ and the coincidence degree before and after data standardization and performs input operation to the operation unit 160 to determine the next processing.

For example, when the coincidence degree before and after data standardization is equal to or smaller than the predetermined threshold set by the user, the user can select whether to recreate the standardized data or exclude the standardized data from the analysis target of the jNMF but carry out the jNMF based on the premise that reproducibility is low.

Furthermore, in a case in which the block ratio of the number of blocks subjected to data complementation or data exclusion to the total is equal to or more than a predetermined threshold set by the user, the user can select whether to change the number of blocks to be aligned in the data set, exclude the blocks from the analysis target of the jNMF, or perform the jNMF on the assumption that reproducibility is low.

It is also possible to select, from a combination of both of the coincidence degree and a ratio of complemented or excluded blocks in all blocks, whether to change the number of blocks to be equalized in a data set, to recreate the standardized data, to exclude the standardized data from the analysis target of the jNMF, or to perform the jNMF based on the premise that reproducibility is low.

A series of these flows is performed in response to input operation of the user to the operation unit 160 but may be automatically performed by, for example, the standardization unit 133A. When the coincidence degree before and after data standardization is equal to or smaller than the predetermined threshold or when the block ratio is equal to or larger than the predetermined threshold, or when the coincidence degree before and after data standardization or the block ratio is equal to or smaller than a predetermined threshold set by the user from a combination of the coincidence degree before and after data standardization or the block ratio, the standardization unit 133A automatically executes the processing explained above.

Note that, when the jNMF is carried out to acquire a clustering result, there is a data set of a known sample, and it is desired to add one sample anew to the data set to perform the jNMF, it is possible to predict a clustering result by applying LSM to markers excluding a marker that does not satisfy a threshold.

FIG. 22 is a flowchart illustrating an example of a flow of coincidence degree calculation processing according to the present embodiment. As illustrated in FIG. 22, in step S81, the standardization unit 133A calculates a coincidence degree between original data and standardized data for each of markers of a sample. In step S82, the display processing unit 134 generates image information including the calculated coincidence degree. In step S83, the display unit 140 displays an image based on the generated image information. Accordingly, the user can visually recognize and grasp the coincidence degree. The user performs input operation to the operation unit 160 and can determine, with reference to the coincidence degree, whether to perform standardization of data again, directly execute analysis, or exclude the data from an analysis target. As explained above, the user is capable of selecting the next processing explained above from the coincidence degree of the data group, the ratio of the number of complemented data in all the data, or both of the coincidence degree and the ratio.

Rather than executing the next processing in response to the input operation of the user to the operation unit 160, the standardization unit 133A may determine, based on the calculated coincidence degree, whether to perform standardization of data again, directly execute the analysis, or exclude the data from the analysis target. For example, when the coincidence degree exceeds the predetermined threshold, the analysis is executed and, when the coincidence degree does not exceed the predetermined threshold, standardization of data is performed again or the data is excluded from the analysis target.

### 1-7. Display Method

### 1-7-1. Display Example of a Tissue Image of a Sample and a Common Module

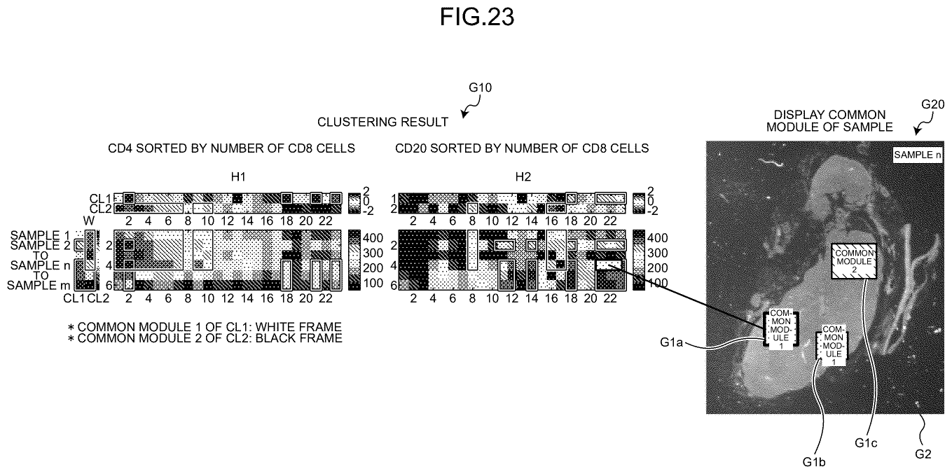

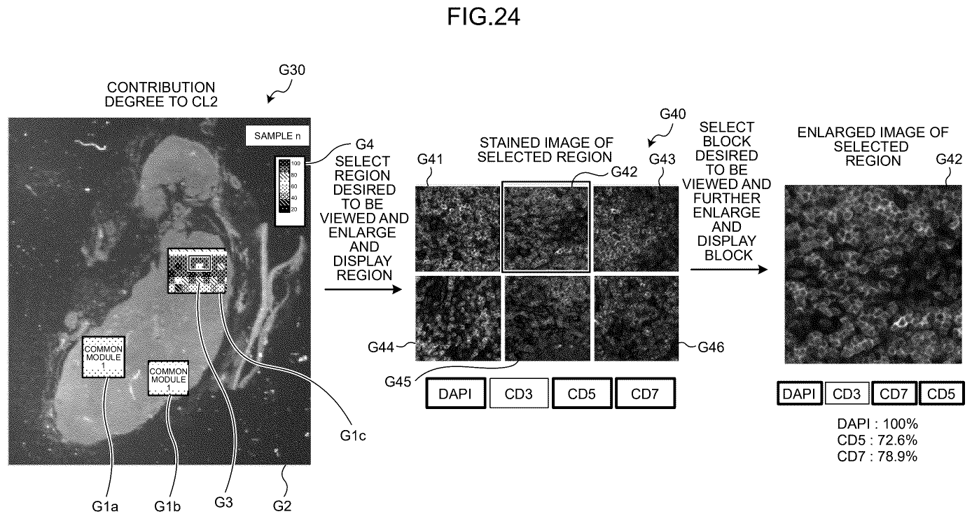

A display example of a tissue image of a sample and a common module according to the present embodiment are explained with reference to FIG. 23 and FIG. 24. FIG. 23 and FIG. 24 are diagrams for explaining examples of display images according to the present embodiment. In the examples illustrated in FIG. 23 and FIG. 24, in a jNMF result, that is, a clustering result, display images in the case in which the user desires to associate a feature region with a pathological image are displayed. The display images are generated by the display processing unit 134.

The display processing unit 134 displays a tissue image of a sample (an example of a specimen image) and a common module based on the clustering result. The common module is a region extracted as membership relating to a clustering result. The membership is a constituent element extracted as a common feature value relating to the clustering result and is, for example, a constituent region (for example, a region or a block) extracted as the common feature value. Here, examples of a flow of display processing by the display processing unit 134 include a flow explained below.

For example, the display processing unit 134 superimposes, based on the clustering result, a common module, which is a region extracted as membership of clusters, on a sample image (for example, a tissue image) and generate a display image. Thereafter, the display processing unit 134 transmits image information concerning the display image to the display unit 140. The display unit 140 displays an image based on the image information transmitted from the display processing unit 134. Note that the display processing unit 134 may generate image information including optimal drug selection and drug effect prediction besides generating image information including an analysis result and image information including a feature value. Since these kinds of image information are displayed by the display unit 140, a user such as a doctor can visually recognize various kinds of information displayed by the display unit 140.

### First Display Example

As illustrated in FIG. 23, the display processing unit 134 executes processing of presenting (displaying) a display image G20 (a figure on the right side in FIG. 23) in association with a block image G10 (a figure on the left side in FIG. 23) indicating the clustering result in a matrix. In the example illustrated in FIG. 23, the clustering result is illustrated based on positional information of a biological sample and a common module of the block image G10 and a common module of the display image G20 are correlated with each other. When the block image G10 is displayed by the display unit 140 and a desired position of the block image G10 is clicked, the display image G20 corresponding to the desired position is displayed by the display unit 140. At this time, the user operates the operation unit 160 to perform click.

Note that the display processing unit 134 superimposes images G1a to G1c indicating regions extracted as the membership of the clusters as a result of the clustering on a sample image (for example, ae tissue image) G2 based on the positional information of the biological sample and generates the display image G20. In the example illustrated in FIG. 23, the regions extracted as the membership of the clusters is described as a common module. It is assumed that a sample n belongs to both of CL1 and CL2 as a result of clustering. CL1 and CL2 indicate classes (clusters). A region allocated as CL1 is described as a common module 1 and a region allocated as CL2 is described as the same common module 2 as CL2 (similarity). Since the display image G20 explained above is displayed by the display unit 140, a user such as a doctor can see and grasp various kinds of information displayed by the display unit 140.

Here, the clustering result in FIG. 23 is an example obtained by setting the number of clusters to 2 and carrying out clustering based on a feature value of a space. The common basis matrix W and the feature vectors H1 and H2 are standardized by a Z score. A part having the Z score higher than a certain cut-off value is allocated as membership of the clusters. In FIG. 23, a part surrounded by a white frame corresponds to the common module 1 allocated as the membership of CL1 and a part surrounded by a black frame corresponds to the common module 2 allocated as the membership of CL2.

In the example illustrated in FIG. 23, a user interface (UI) that associates where in a sample image (common module display of a sample) a mass of common modules of a clustering result is located. For example, when a region of the mass is clicked, the display image G20 is displayed such that it is seen which region of the sample image G2 the region corresponds to. As explained above, it is possible to check, from the result of the clustering, which region of the sample image the region extracted as the common module corresponds to. For example, the region extracted as the common module 1 is divided into two regions and described in the display image G20. However, the correspondence display illustrated in FIG. 23 is convenient, for example, when checking which block of the common module 1 of the clustering result one region of the two regions of the common module 1 is equivalent to.

### Second Display Example

As illustrated in FIG. 24, the display processing unit 134 generates a heat map G3 indicating a degree of contribution to each of the clusters of the region for each of the common modules and superimposes the generated heat map G3 on the sample image G2 based on the positional information of the common module and generates a display image G30. The display image G30 is displayed by the display unit 140. In the example illustrated in FIG. 24, the heat map G3 indicating the degree of contribution to the cluster (CL2) of the region is superimposed on the common module 2 (the image G1c in FIG. 24) of the sample images. Note that a color bar G4 relating to the heat map G3 is also superimposed on the sample image G2.

The display processing unit 134 executes processing of presenting a stained image G40 corresponding to the common module according to selection of the common module for the display image G30. For example, when a region desired to be viewed is selected in the common module of the display image G30 in FIG. 24, stained images G41 to G46 for each staining markers corresponding to the selected region are displayed. At this time, the user operates the operation unit 160 to select a region. When a desired stained image G42 is clicked out of the stained images G41 to G46 for each of the staining markers, the clicked stained image G42 is enlarged and displayed. Furthermore, when a plurality of stained images among the stained images G41 to G46 are clicked and selected, superimposed display of the staining markers is implemented. At this time, the user operates the operation unit 160 to perform click.

In the example illustrated in FIG. 24, when the user designates a region of interest (a portion surrounded by a black frame in the common module), the user can view a stained image of the region. The superimposition of the staining markers can be switched by turning on/off buttons of DAPI, CD3, CD5, and CD7. Colors of the buttons may be set the same as colors in images of the staining markers. For example, DAPI is indicated by blue, CD3 is indicated by yellowish green, CD5 is indicated by red, and CD7 is indicated by light blue. Furthermore, when the user desires to perform enlarged display, the user can perform further enlarged display by selecting a block desired to be viewed (the portion surrounded by the black frame). The user can check positive cell rates and the numbers of positive cells of the staining markers in the selected region.

As explained above, according to the second display example, when the user selects a region that the user desires to view, the region is switched to a stained image. It is possible to perform enlarged display of the stained image and superimposed display of the staining markers used for analysis. It is possible to display positive cell rates and the numbers of positive cells of the staining markers for each of blocks and it is possible to check various kinds of information for each of the blocks.

### 1-7-2. Display Example of jNMF Results

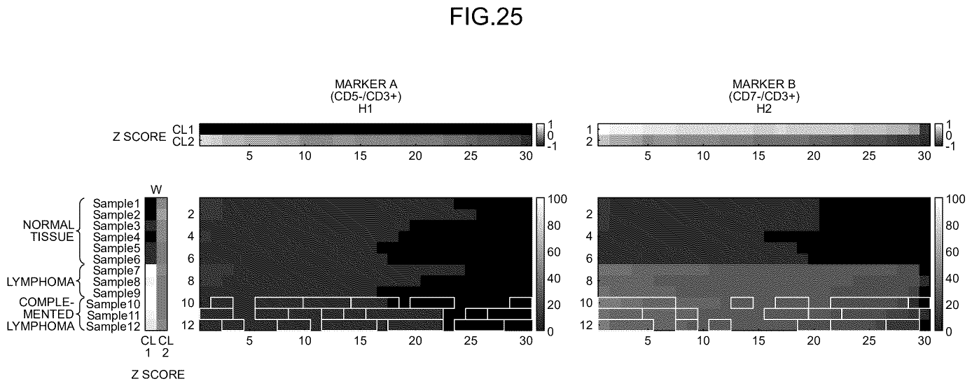

A display example of jNMF results according to the present embodiment is explained with reference to FIG. 25 to FIG. 31. FIG. 25 to FIG. 31 are respectively diagrams for explaining examples of display images according to the present embodiment. In the examples illustrated in FIG. 25 to FIG. 31, jNMF results are displayed.

In the first display example and the second display example explained above, when the data to be displayed is complemented data (complementary data), there is no correspondence with an actual image (the sample image G2) and it is likely that there is no image that can be displayed. In order to avoid this, as illustrated in FIG. 25, the display processing unit 124 performs grouping such that actual data closest to the complementary data is included. In the example illustrated in FIG. 25, a white frame indicates a grouped region. Specifically, the complementary data is grouped into actual data having the closest value. For example, when there is actual data having the same value, the complementary data is grouped with data of lower order at the time when being sorted by the reference marker. For example, blocks adjacent to one another are grouped such that one actual data closest to the complementary data is included.

Accordingly, when a region pointed by the user is the complementary data, the complementary data jumps to actual data having the closest value to the complementary data and the actual data is displayed. Even if the user selects the complementary data while being able to understand how large region has been subjected to data complementation, since an image region of the grouped closest actual data is displayed, it is possible to prevent an image that can be displayed from becoming absent as explained above.

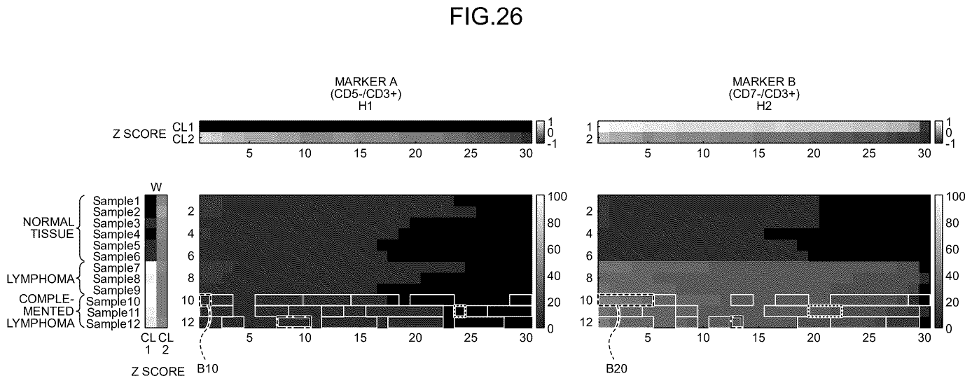

Since both of the marker A and the marker B are sorted by the reference marker, blocks located in the same order in the marker A and the marker B are the same block on an image. For example, as illustrated in FIG. 26, a first block B10 of the marker A and a first block B20 of the marker B of a Sample 10 are the same region on the image. When the grouped pieces of data are regarded as one group and the order of the pieces of data is counted, since the position of the actual data can be known, a correspondence relationship between the blocks of the marker A and the marker B can be easily understood. Note that, together with the result display of the jNMF, a complementation ratio of the data may be indicated for each of samples.

### Other Display Examples

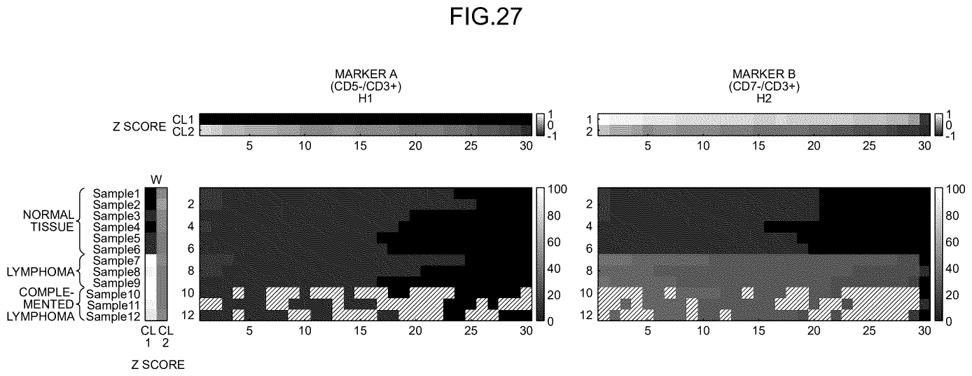

As illustrated in FIG. 27, the display processing unit 134 may highlight a region of the complementary data with a mode such as filling by hatching or shading, graying out, or translucency such that the complementary data can be visually recognized. In the example illustrated in FIG. 27, the filling by the hatching is executed. Note that, together with the result display of the jNMF, a complementation ratio of the data may be indicated for each of samples. When the complementary data and the actual data are not grouped, if the user selects the complementary data, the display processing unit 134 may execute measures explained below according to the selection.

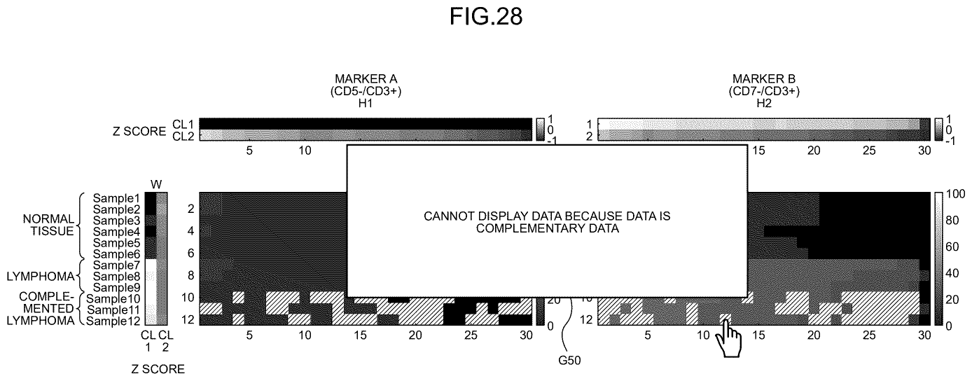

For example, as illustrated in FIG. 28, when the user selects the complementary data with clicking or the like, the display processing unit 134 executes display of an error image G50 including a sentence such as “Because the data is complementary data, the data cannot be displayed”. Specifically, the display processing unit 134 generates image information including the error image G50 and the display unit 140 displays an image including the error image G50 based on the image information. Rather than the display of the error image G50, the display processing unit 134 may execute, for example, a measure of not displaying the next even if the next image is selected.

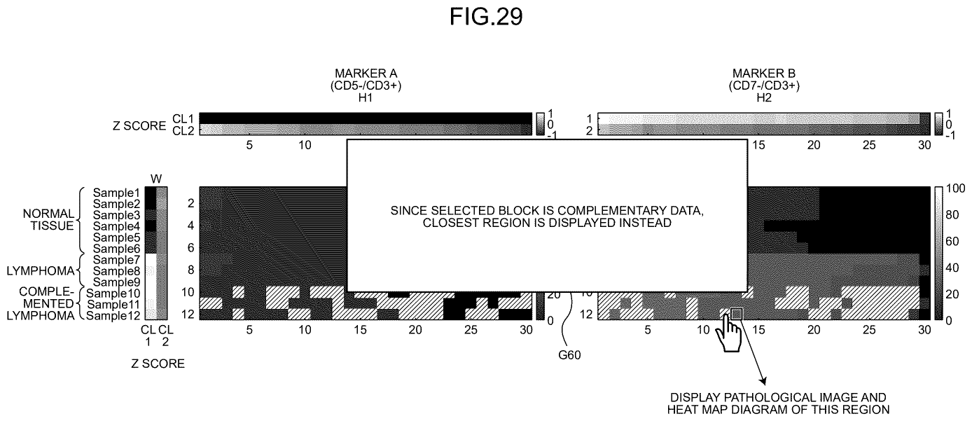

As illustrated in FIG. 29, when the user selects the complementary data with clicking or the like, the display processing unit 134 executes display of an error image G60 including a sentence such as “Because the selected block is a complementary data, the closest region is displayed instead”. Specifically, the display processing unit 134 generates image information including the error image G60 and the display unit 140 displays an image including the error image G60 based on the image information. Furthermore, the display processing unit 134 executes display of an image such as a pathological image or a heat map of a region closest to the selected block. That is, when the user selects a block in which data is complemented, the display processing unit 134 causes the display unit 140 to display a result of the closest block after error display.

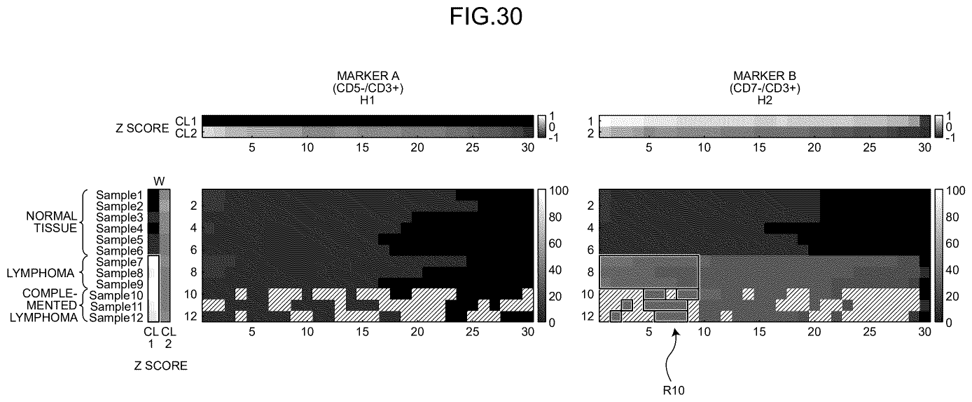

As illustrated in FIG. 30, for example, when displaying the common module (see FIG. 23) with the result of the jNMF, the display processing unit 134 generates, for the common module, image information not including complementary data and executes display. This is to display correspondence of a position to a pathological image, a cluster contribution degree, features of a space, and the like from the common module. In the example illustrated in FIG. 30, a region R10 surrounded by black frames of the marker B is a common module region of the lymphoma Samples 7 to 12.

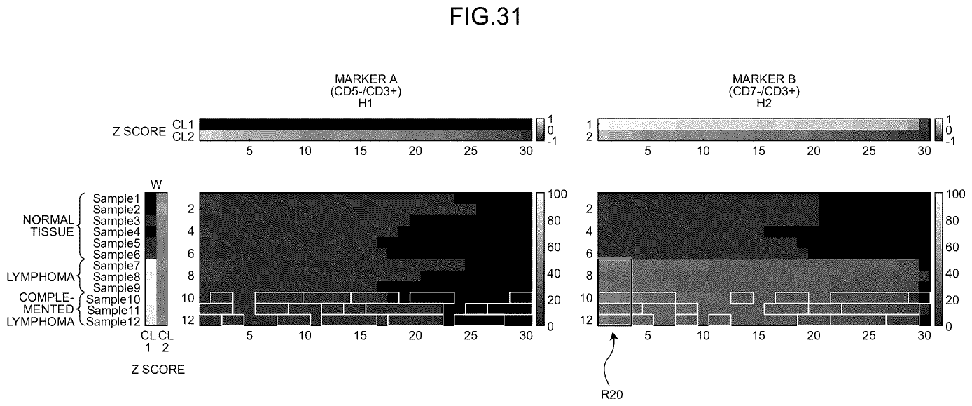

Note that, as illustrated in FIG. 31, when a common module is extracted, grouped regions sometimes extend across the common module. In the example illustrated in FIG. 31, a region R20 surrounded by a black frame of the marker B is a common module region of the lymphoma Samples 7 to 12.

For example, when actual data is included in the common module, the display processing unit 134 leaves the grouped regions extending across the common module. In addition, when a part of saved data is included in the common module and actual data is not included in the common module, the display processing unit 134 enables the user to select whether to include the actual data in the common module. For example, the display processing unit 134 provides a UI for selection.

### 1-8. Other Examples of the Data Complementation Method

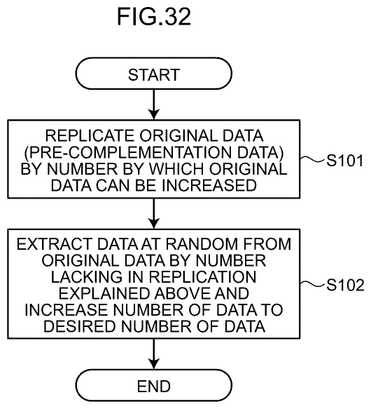

Other examples of the data complementation method according to the present embodiment are explained with reference to FIG. 32 to FIG. 34. As the data complementation method, besides the method explained above of complementing the lacking number of pieces of data based on the average value μ and the standard deviation σ of the pre-complementation data, there is a method of replicating and complementing data by a lacking number out of the pre-complementation data.

FIG. 32 is a flowchart illustrating an example of a flow of the data complementation processing according to the present embodiment. As illustrated in FIG. 32, in step S101, the standardization unit 133A replicates (doubles) original data (pre-complementation data) by a number by which the original data can be increased. In step S102, the standardization unit 133A extracts data from the original data at random by a number lacking in the replication explained above and increases the number of pieces of data to a desired number of pieces of data (the number of pieces of data desired to be equalized). As explained above, it is possible to replicate and complement the data by the lacking number out of the pre-complementation data. Note that this method is a data complementation method examined using actual specimen data rather than simulation data.

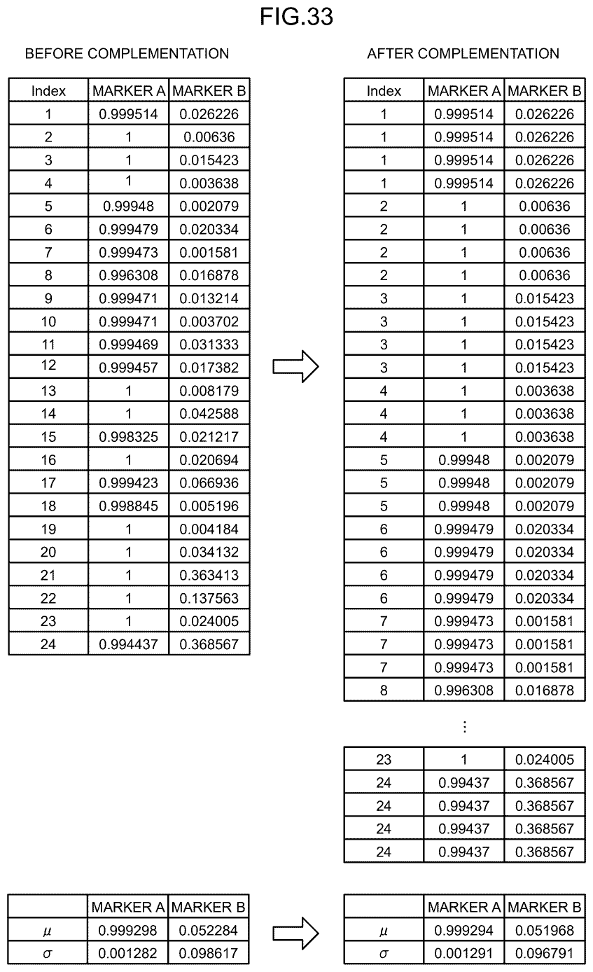

FIG. 33 is a diagram for explaining an example of the data complementation processing according to the present embodiment. As illustrated in FIG. 33, for example, when the number of pieces of data (the number of block regions) of original data of a Sample A is twenty-four and the number of pieces of data after complementation of the Sample A is increased to eighty-eight, it is necessary to increase the number of pieces of data by three times or more. In the example illustrated in FIG. 33, the marker A is set as the number of CD3+CD5+ cells/the number of CD3+ cells and the marker B is set as the number of CD3+CD7+ cells/the number of CD3+ cells.

The standardization unit 133A replicates the original data to three times (24×3=72) first and increases lacking sixteen pieces of missing data at random from the original data. In the example illustrated in FIG. 33, the standardization unit 133A calculates the marker A and the marker B (the positive cell rate) for each of block regions, sorts the block regions with the number of CD3+ cells, and allocates index numbers in the descending order. In the example illustrated in FIG. 33, data in which one or more index numbers overlap is replicated data and the data is replicated such that there are three or four same index numbers. These replication numbers are set as predetermined values in advance but may be changeable by input operation of the user to the operation unit 160.

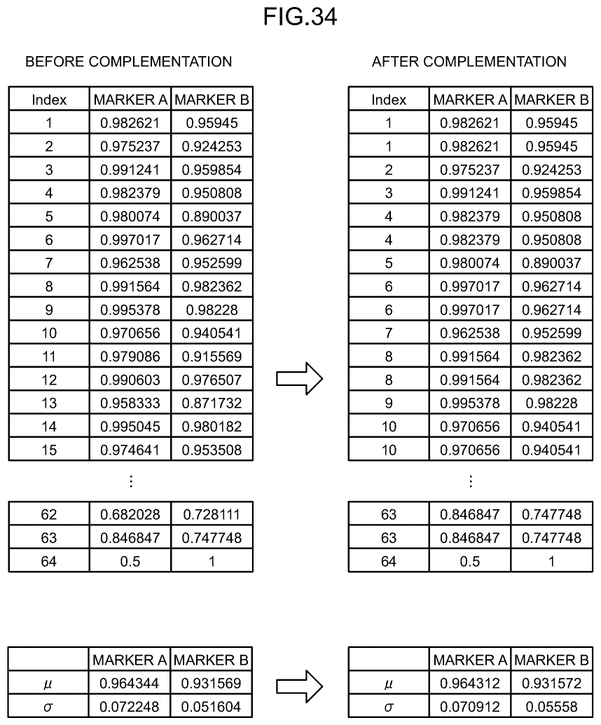

FIG. 34 is a diagram for explaining an example of the data complementation processing according to the present embodiment. As illustrated in FIG. 34, for example, when the number of o pieces of data (the number of block regions) of original data of a Sample B is sixty-four and the number of pieces of data after complementation of the Sample B is increased to eighty-eight, it is necessary to increase the number of pieces of data by slightly less than 1.5 times. In the example illustrated in FIG. 34, as in FIG. 33, the marker A is set as the number of CD3+CD5+ cells/the number of CD3+ cells and the marker B is set as the number of CD3+CD7+ cells/the number of CD3+ cells.

Since the original data cannot be entirely replicated, the standardization unit 133A increases the lacking twenty-four pieces of data at random from the original data. Note that, in the example illustrated in FIG. 34, as in FIG. 33, the standardization unit 133A calculates the marker A and the marker B (the positive cell rate) for each of the block regions, sorts the block regions with the number of CD3+ cells, and allocates index numbers in the descending order. In the example illustrated in FIG. 34, data in which one or more index numbers overlap is replicated data and the data is replicated at random.

As illustrated in FIG. 33 and FIG. 34, since the average values μ and the standard deviations σ of the markers A and B of each of the data set are substantially the same before the complementation and after the complementation, a result of the jNMF is not affected. Since the complemented data is always set with the actual data, in the case of the result display of the jNMF, the complemented data and the actual data can be encircled and displayed as illustrated in FIG. 31. Since the complemented data is generated by directly replicating the original data, even when the user selects the complemented data, it is unnecessary to perform error display as illustrated in FIG. 28 and FIG. 29 and an image can be presented to the user as illustrated in FIG. 23.

With data complementation method explained above, it is possible to complement data with the set of the marker A and the marker B. Therefore, it is possible to directly replicate a predetermined region space. Since the original data is present in all the data at the time of the jNMF result display (there is no case in which the original data is absent), it is possible to present result display and a relevant place of an image.

### 1-9. Action and Effects

As described above, according to the present embodiment, the information processing device 100 includes the acquisition unit 110 that acquires a fluorescence spectrum deriving from a biological sample and positional information of the biological sample from a sample including the biological sample, the specifying unit 133b that specifies, from the fluorescence spectrum, pieces of information concerning a plurality of different biomarkers of the biological sample associated with the positional information of the biological sample, the standardization unit 133A that performs information complementation or exclusion with respect to the pieces of information concerning the plurality of biomarkers, and the correlation analysis unit 133d that performs matrix decomposition processing corresponding to a combination of the plurality of biomarkers with respect to the pieces of information concerning the plurality of biomarkers for which the information complementation or exclusion has been performed, and outputs a correlation among the pieces of information concerning the plurality of biomarkers. Accordingly, it is possible to accurately acquire the correlation among the pieces of information concerning the plurality of biomarkers. Therefore, it is possible to accurately obtain a correlation among the plurality of biomarkers.

The correlation analysis unit 133d may execute, with the jNMF, the matrix decomposition processing on the pieces of information concerning the plurality of biomarkers for which information complementation or exclusion has been performed. Accordingly, it is possible to accurately and securely obtain the correlation among the plurality of biomarkers.

The standardization unit 133A may calculate the average value μ and the standard deviation σ of the pieces of information concerning the plurality of biomarkers, generate random numbers having a normal distribution that satisfies the calculated average value μ and the calculated standard deviation σ, and generate information complementing the pieces of information concerning the plurality of biomarkers. Accordingly, it is possible to accurately and securely obtain the correlation among the plurality of biomarkers.

The standardization unit 133A may replicate a part of the pieces of information concerning the plurality of biomarkers and generate information that complements the pieces of information concerning the plurality of biomarkers. Accordingly, it is possible to accurately and securely obtain the correlation among the plurality of biomarkers.

The standardization unit 1331A may exclude, from the pieces of information concerning the plurality of biomarkers, one or both of information concerning a region without a tissue and information concerning a region where a luminance value is equal to or larger than a predetermined threshold. Accordingly, it is possible to accurately and securely obtain the correlation among the plurality of biomarkers.

The information processing device 100 may further include the sorting unit 133c that changes, based on the arrangement order of a plurality of pieces of unit information (for example, blocks) included in information concerning one biomarker among pieces of information concerning a plurality of biomarkers for which information complementation or exclusion has been executed, the arrangement order of a plurality of pieces of unit information (for example, blocks) included in information concerning the other biomarkers. The correlation analysis unit 133d may perform matrix decomposition processing on the pieces of information concerning the plurality of biomarkers, the arrangement order of which has been changed. Accordingly, it is possible to accurately and securely obtain the correlation among the plurality of biomarkers.

The standardization unit 133A may calculate a coincidence degree between the pieces of information concerning the plurality of biomarkers and the pieces of information concerning the plurality of biomarkers for which the information complementation or exclusion has been performed. Accordingly, it is possible to obtain a coincidence degree between original data before the complementation and data after the complementation.

The information processing device 100 may further include the display processing unit 134 that generates image information including the calculated coincidence degree. Accordingly, it is possible to obtain image information including the coincidence degree between the original data before the complementation and the data after the complementation.

The standardization unit 133A may repeat the information complementation or exclusion according to the calculated coincidence degree. Accordingly, it is possible to increase the coincidence degree between the original data before the complementation and the data after the complementation.

The information processing device 100 may further include the display processing unit 134 that generates image information including the correlation among the pieces of information concerning the plurality of biomarkers. Accordingly, it possible to obtain image information including the correlation among the pieces of information concerning the plurality of biomarkers.

When complementation of information is executed, the display processing unit 134 may generate image information to group and display the complemented information and actual information closest to the complemented information (see FIG. 25 and FIG. 26). Accordingly, it is possible to obtain image information including the grouping concerning the complementation of the information.

When the complementation of the information is executed, the display processing unit 134 may generate image information to highlight the complemented information (see FIG. 27). Accordingly, it is possible to obtain image information including the highlighting concerning the complementation of the information.

When the complemented information is selected according to information selection for the image information, the display processing unit 134 may generate error image information indicating that the complemented information has been selected (see FIG. 28 and FIG. 29). Accordingly, it is possible to obtain error image information including error display concerning the complementation of the information.

The display processing unit 134 may generate image information concerning actual information closest to the complemented information according to the information selection for the image information (see FIG. 29). Accordingly, it is possible to obtain the image information concerning the actual information closest to the complemented information.

When the complementation of the information is executed, the display processing unit 134 may generate image information not including the complemented information (see FIG. 30). Accordingly, it is possible to obtain image information not including the complemented information.

The information processing device 100 may further include the selection unit 133a that determines a predetermined region of a sample. The specifying unit 133b may specify, from a fluorescence spectrum of the predetermined region, information concerning a plurality of biomarkers linked with positional information of a biological sample in the predetermined region. Accordingly, it is possible to obtain a correlation among biomarkers in the predetermined region (for example, a region of interest) of the sample.

The predetermined region may be set by the user. Accordingly, it is possible to set a predetermined region desired by the user. Therefore, it is possible to obtain a correlation among biomarkers in the predetermined region corresponding to the desire of the user.

The selection unit 133a may determine individual predetermined region of a plurality of the samples, the acquisition unit 110 may acquire a fluorescence spectrum and positional information of a biological sample for each of predetermined regions, the specifying unit 133b may specify, from the fluorescence spectrum for each of the predetermined regions, information concerning a plurality of biomarkers for each of predetermined regions linked with the positional information of the biological sample for each of predetermined regions, the standardization unit 133A may execute information complementation or exclusion on the pieces of information concerning the plurality of biomarkers for each of the predetermined regions, and the correlation analysis unit 133d may perform matrix decomposition processing on the pieces of information concerning the plurality of biomarkers for each of the predetermined regions for which the information complementation or exclusion has been executed. Accordingly, it is possible to obtain a correlation among biomarkers in individual predetermined regions of a plurality of samples.

The standardization unit 133A may change processing according to the calculated coincidence degree. Accordingly, it is possible to implement processing corresponding to a coincidence degree between original data before complementation and data after the complementation.

The plurality of samples may be a plurality of different specimens. Accordingly, it is possible to obtain a correlation among biomarkers in a plurality of different specimens.

### 2. OTHER EMBODIMENTS

The configurations, the processing, and the like according to the embodiment (including the examples and the modifications) may be implemented in various different modes other than the embodiment explained above. For example, among the types of processing explained in the embodiments, all or a part of the types of processing explained as being automatically performed can be manually performed or all or a part of the types of processing explained as being manually performed can be automatically performed by a known method. Besides, the processing procedures, the specific names, and the information including the various data and parameters explained in the document and illustrated in the drawings can be optionally changed except when specifically noted otherwise. For example, the various kinds of information illustrated in the figures are not limited to the illustrated information.

The configurations, the processing, and the like according to the embodiment explained above (including the examples and the modifications) do not always need to be physically configured as illustrated in the figures. That is, specific forms of distribution and integration of the devices are not limited to the illustrated forms and all or a part thereof can be configured by being functionally or physically distributed and integrated in any unit according to various loads, usage situations, and the like.

The configurations, the processing, and the like according to the embodiment explained above (including the examples and the modifications) may be combined as appropriate. For example, at least a part of the embodiment may be combined as appropriate with at least a part of other embodiments. The effects in the embodiment are only exemplifications and are not limited, and other effects may be present.

### 3. APPLIED EXAMPLES

The technology according to the present disclosure can be applied to, for example, a fluorescence observation apparatus 500 or the like which is an example of a microscope system.

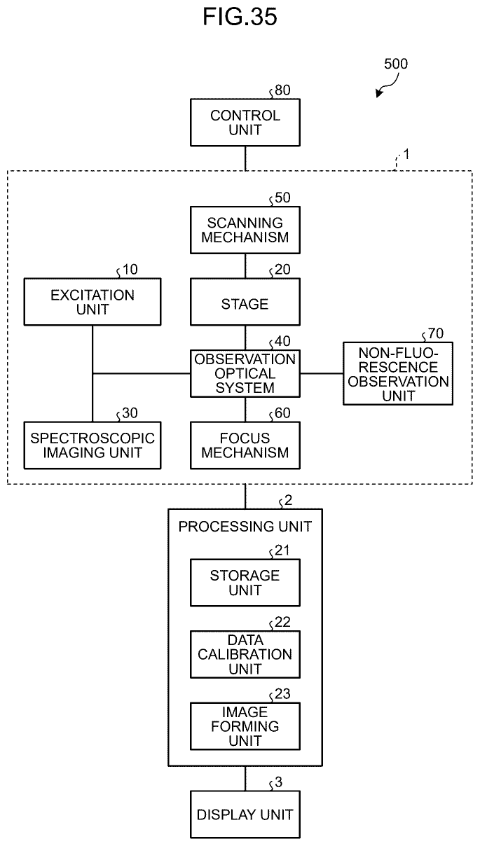

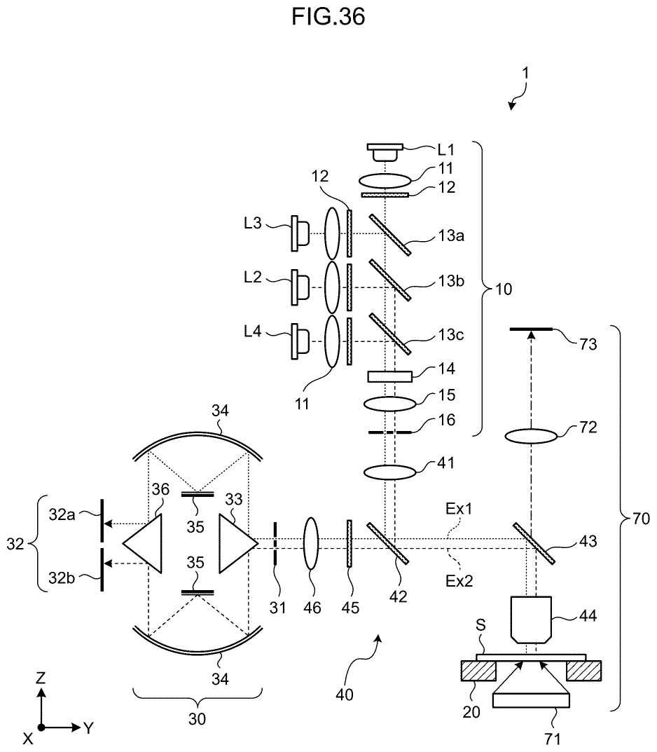

Hereinafter, a configuration example of an applicable fluorescence observation apparatus 500 will be described with reference to FIGS. 35 and 36. FIG. 35 is a diagram showing an example of a schematic configuration of the fluorescence observation apparatus 500 according to the present embodiment. FIG. 36 is a diagram showing an example of a schematic configuration of an observation unit 1 according to the present embodiment.

As shown in FIG. 35, the fluorescence observation apparatus 500 includes the observation unit 1, a processing unit 2, and a display unit 3.

The observation unit 1 includes an excitation unit (irradiation unit) 10, a stage 20, a spectroscopic imaging unit 30, an observation optical system 40, a scanning mechanism 50, a focus mechanism 60, and a non-fluorescence observation unit 70.

The excitation unit 10 irradiates the observation target with a plurality of beams of irradiation light having different wavelengths. For example, the excitation unit 10 irradiates a pathological specimen, that is a pathological sample, which is the observation target, with a plurality of line illuminations having different wavelengths arranged in parallel with different axes. The stage 20 is a table that supports the pathological specimen, and is configured to be movable in a direction perpendicular to the direction of line light by the line illuminations by the scanning mechanism 50. The spectroscopic imaging unit 30 includes a spectroscope and acquires a fluorescence spectrum, that is spectroscopic data, of the pathological specimen excited linearly by the line illuminations.

That is, the observation unit 1 functions as a line spectroscope that acquires spectroscopic data corresponding to the line illuminations. Further, the observation unit 1 also functions as an imaging device that captures a plurality of fluorescence images generated by the pathological specimen, which is an imaging target, for each of a plurality of fluorescence wavelengths for each line and acquires data of the plurality of captured fluorescence images in an arrangement order of the lines.

Here, parallel with different axis means that the plurality of line illuminations has different axes and are parallel. The different axes mean that the axes are not coaxial, and the distance between the axes is not particularly limited. The parallel is not limited to parallel in a strict sense, and includes a state of being substantially parallel. For example, there may be distortion originated from an optical system such as a lens or deviation from a parallel state due to manufacturing tolerance, and this case is also regarded as parallel.

The excitation unit 10 and the spectroscopic imaging unit 30 are connected to the stage 20 via the observation optical system 40. The observation optical system 40 has a function of following an optimum focus by the focus mechanism 60. The non-fluorescence observation unit 70 for performing dark field observation, bright field observation, and the like may be connected to the observation optical system 40. In addition, a control unit 80 that controls the excitation unit 10, the spectroscopic imaging unit 30, the scanning mechanism 50, the focus mechanism 60, the non-fluorescence observation unit 70, and the like may be connected to the observation unit 1.

The processing unit 2 includes a storage unit 21, a data calibration unit 22, and an image forming unit 23. The processing unit 2 typically forms an image of the pathological specimen or outputs a distribution of the fluorescence spectrum on the basis of the fluorescence spectrum of the pathological specimen acquired by the observation unit 1. Hereinafter, the pathological specimen is also referred to as a sample S. The image herein refers to a constituent ratio of autofluorescence derived from a dye or a sample, or the like constituting the spectrum, an image converted from waveforms into RGB (red, green, and blue) color, a luminance distribution in a specific wavelength band, and the like.

The storage unit 21 includes a nonvolatile storage medium such as a hard disk drive or a flash memory, and a storage control unit that controls writing and reading of data to and from the storage medium. The storage unit 21 stores spectroscopic data indicating a correlation between each wavelength of light emitted by each of the plurality of line illuminations included in the excitation unit 10 and fluorescence received by the camera of the spectroscopic imaging unit 30. Further, the storage unit 21 stores in advance information indicating a standard spectrum of autofluorescence related to a sample (pathological specimen) to be observed and information indicating a standard spectrum of a single dye staining the sample.

The data calibration unit 22 configures the spectroscopic data stored in the storage unit 21 on the basis of the captured image captured by the camera of the spectroscopic imaging unit 30. The image forming unit 23 forms a fluorescence image of the sample on the basis of the spectroscopic data and an interval Δy of the plurality of line illuminations irradiated by the excitation unit 10. For example, the processing unit 2 including the data calibration unit 22, the image forming unit 23, and the like is implemented by hardware elements used in a computer such as a central processing unit (CPU), a random access memory (RAM), and a read only memory (ROM), and a necessary program (software). Instead of or in addition to the CPU, a programmable logic device (PLD) such as a field programmable gate array (FPGA), a digital signal processor (DSP), an application specific integrated circuit (ASIC), or the like may be used.

The display unit 3 displays, for example, various types of information such as an image based on the fluorescence image formed by the image forming unit 23. The display unit 3 may include, for example, a monitor integrally attached to the processing unit 2, or may be a display device connected to the processing unit 2. The display unit 3 includes, for example, a display element such as a liquid crystal device or an organic EL device, and a touch sensor, and is configured as a user interface (UI) that displays input settings of image-capturing conditions, a captured image, and the like.

Next, details of the observation unit 1 will be described with reference to FIG. 36. Here, a description will be given on the assumption that the excitation unit 10 includes two line illuminations Ex1 and Ex2 that each emit light of two wavelengths. For example, the line illumination Ex1 emits light having a wavelength of 405 nm and light having a wavelength of 561 nm, and the line illumination Ex2 emits light having a wavelength of 488 nm and light having a wavelength of 645 nm.

As shown in FIG. 36, the excitation unit 10 includes a plurality of excitation light sources L1, L2, L3, and L4. Each of the excitation light sources L1 to L4 includes a laser light source that outputs laser light having a wavelength of 405 nm, 488 nm, 561 nm, and 645 nm, respectively. For example, each of the excitation light sources L1 to L4 includes a light emitting diode (LED), a laser diode (LD), or the like.

Furthermore, the excitation unit 10 includes a plurality of collimator lenses 11, a plurality of laser line filters 12, a plurality of dichroic mirrors 13a, 13b, and 13c, a homogenizer 14, a condenser lens 15, and an incident slit 16 so as to correspond to each of the excitation light sources L1 to L4.

The laser light emitted from the excitation light source L1 and the laser light emitted from the excitation light source L3 are collimated by the collimator lens 11, transmitted through the laser line filter 12 for cutting a skirt of each wavelength band, and made coaxial by the dichroic mirror 13a. The two coaxial laser lights are further beam-shaped by the homogenizer 14 such as a fly-eye lens and the condenser lens 15 so as to be the line illumination Ex1.

Similarly, the laser light emitted from the pumping light source L2 and the laser light emitted from the excitation light source L4 are coaxial by the dichroic mirrors 13b and 13c, and line illumination is performed so that the line illumination Ex2 is different in axis from the line illumination Ex1. The line illuminations Ex1 and Ex2 form line illuminations with different axes, that is primary image, which are separated by a distance Δy in the incident slit 16 having a plurality of slit portions through which each of the line illuminations Ex1 and Ex2 can pass.

Note that, in the present embodiment, an example in which the four lasers have two coaxial axes and two different axes will be described, but in addition to this, the two lasers may have two different axes or the four lasers may have four different axes.

The sample S on the stage 20 is irradiated with the primary image via the observation optical system 40. The observation optical system 40 includes a condenser lens 41, dichroic mirrors 42 and 43, an objective lens 44, a band pass filter 45, and a condenser lens 46. The condenser lens 46 is an example of an imaging lens. The line illuminations Ex1 and Ex2 are collimated by the condenser lens 41 paired with the objective lens 44, reflected by the dichroic mirrors 42 and 43, transmitted through the objective lens 44, and irradiates the sample S on the stage 20.

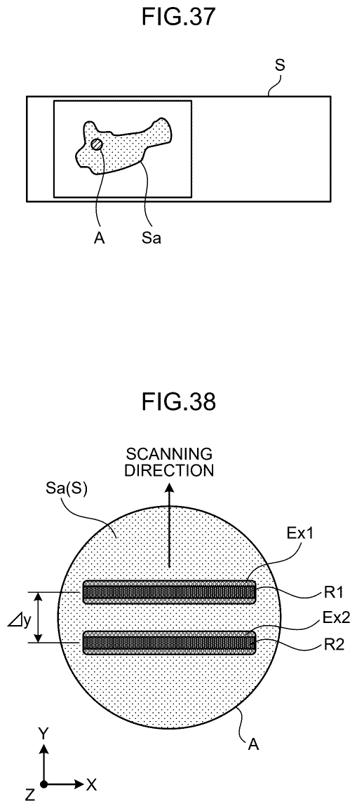

Here, FIG. 37 is a diagram showing an example of the sample S according to the present embodiment. FIG. 37 shows a state in which the sample S is viewed from the irradiation directions of the line illuminations Ex1 and Ex2 as excitation light. The sample S is typically configured by a slide including an observation target Sa such as a tissue section as shown in FIG. 37, but may be of course other than that. The observation target Sa is, for example, a biological sample such as a nucleic acid, a cell, a protein, a bacterium, or a virus. The sample S, that is, observation target Sa is stained with a plurality of fluorescent dyes. The observation unit 1 enlarges and observes the sample S at a desired magnification.

FIG. 38 is an enlarged diagram showing a region A in which the sample S according to the present embodiment is irradiated with the line illuminations Ex1 and Ex2. In the example of FIG. 38, two line illuminations Ex1 and Ex2 are arranged in the region A, and imaging areas R1 and R2 of the spectroscopic imaging unit 30 are arranged so as to overlap the line illuminations Ex1 and Ex2. The two line illuminations Ex1 and Ex2 are each parallel to a Z-axis direction and are arranged apart from each other by a predetermined distance Δy in a Y-axis direction.

The line illuminations Ex1 and Ex2 are formed on the surface of the sample S as shown in FIG. 38. As shown in FIG. 36, fluorescence excited in the sample S by the line illuminations Ex1 and Ex2 is condensed by the objective lens 44, reflected by the dichroic mirror 43, transmitted through the dichroic mirror 42 and the band pass filter 45 that cuts off the excitation light, condensed again by the condenser lens 46, and incident on the spectroscopic imaging unit 30.

As shown in FIG. 36, the spectroscopic imaging unit 30 includes an observation slit 31, an imaging element 32, a first prism 33, a mirror 34, a diffraction grating 35, and a second prism 36. The observation slit 31 is an opening. The diffraction grating 35 is, for example, a wavelength dispersion element.

In the example of FIG. 36, the imaging element 32 includes two imaging elements 32a and 32b. The imaging element 32 receives a plurality of light beams, for example fluorescence and the like, wavelength-dispersed by the diffraction grating 35. As the imaging element 32, for example, a two-dimensional imager such as a charge coupled device (CCD) or a complementary metal oxide semiconductor (CMOS) is employed.

The observation slit 31 is disposed at the condensing point of the condenser lens 46, and has the same number of (two this example) slit portions as the number of excitation lines. The fluorescence spectra derived from the two excitation lines that have passed through the observation slit 31 are separated by the first prism 33 and reflected by a grating surface of the diffraction grating 35 via the mirror 34, so that the fluorescence spectra are further separated into fluorescence spectra of respective excitation wavelengths. The four separated fluorescence spectra are incident on the imaging elements 32a and 32b via the mirror 34 and the second prism 36, and are developed as spectroscopic data into spectroscopic data (x, λ) expressed by the position x in the line direction and the wavelength λ. The spectroscopic data (x, λ) is a pixel value of a pixel at a position x in a row direction and at a position of a wavelength λ in a column direction among pixels included in the imaging element 32. Note that the spectroscopic data (x, λ) may be simply described as spectroscopic data.

Note that the pixel size (nm/Pixel) of the imaging elements 32a and 32b is not particularly limited, and is set, for example, equal to or more than 2 (nm/Pixel) and equal to or less than 20 (nm/Pixel). This dispersion value may be achieved optically or at a pitch of the diffraction grating 35, or may be achieved by using hardware binning of the imaging elements 32a and 32b. In addition, the dichroic mirror 42 and the band pass filter 45 are inserted in the middle of the optical path so that the excitation light, that is, line illuminations Ex1 and Ex2 does not reach the imaging element 32.

Each of the line illuminations Ex1 and Ex2 is not limited to the case of being configured with a single wavelength, and each may be configured with a plurality of wavelengths. When the line illuminations Ex1 and Ex2 are each formed by a plurality of wavelengths, the fluorescence excited by these also includes a plurality of spectra. In this case, the spectroscopic imaging unit 30 includes a wavelength dispersion element for separating the fluorescence into a spectrum derived from the excitation wavelength. The wavelength dispersion element includes a diffraction grating, a prism, or the like, and is typically disposed on an optical path between the observation slit 31 and the imaging element 32.

Note that the stage 20 and the scanning mechanism 50 constitute an X-Y stage, and move the sample S in the X-axis direction and the Y-axis direction in order to acquire a fluorescence image of the sample S. In the whole slide imaging (WSI), an operation of scanning the sample S in the Y-axis direction, then moving the sample S in the X-axis direction, and further performing scanning in the Y-axis direction is repeated. By using the scanning mechanism 50, it is possible to continuously acquire dye spectra, that is fluorescence spectra, excited at different excitation wavelengths, which are spatially separated by the distance Δy on the sample S, that is observation target Sa, in the Y-axis direction.

The scanning mechanism 50 changes the position irradiated with the irradiation light in the sample S over time. For example, the scanning mechanism 50 scans the stage 20 in the Y-axis direction. The scanning mechanism 50 can cause the stage 20 to scan the plurality of line illuminations Ex1 and Ex2 in the Y-axis direction, that is, in the arrangement direction of the line illuminations Ex1 and Ex2. This is not limited to this example, and the plurality of line illuminations Ex1 and Ex2 may be scanned in the Y-axis direction by a galvano mirror disposed in the middle of the optical system. Since the data derived from each of the line illuminations Ex1 and Ex2 (for example, the two-dimensional data or the three-dimensional data) is data whose coordinates are shifted by the distance Δy with respect to the Y axis, the data is corrected and output on the basis of the distance Δy stored in advance or the value of the distance Δy calculated from the output of the imaging element 32.

As shown in FIG. 36, the non-fluorescence observation unit 70 includes a light source 71, the dichroic mirror 43, the objective lens 44, a condenser lens 72, an imaging element 73, and the like. In the non-fluorescence observation unit 70, an observation system by dark field illumination is shown in the example of FIG. 36.

The light source 71 is disposed on the side facing the objective lens 44 with respect to the stage 20, and irradiates the sample S on the stage 20 with illumination light from the side opposite to the line illuminations Ex1 and Ex2. In a case of the dark field illumination, the light source 71 illuminates from the outside of the NA (numerical aperture) of the objective lens 44, and light (dark field image) diffracted by the sample S is imaged by the imaging element 73 via the objective lens 44, the dichroic mirror 43, and the condenser lens 72. By using dark field illumination, even a apparently transparent sample such as a fluorescently-stained sample can be observed with contrast.

Note that this dark field image may be observed simultaneously with fluorescence and used for real-time focusing. In this case, as the illumination wavelength, a wavelength that does not affect fluorescence observation may be selected. The non-fluorescence observation unit 70 is not limited to the observation system that acquires a dark field image, and may be configured by an observation system that can acquire a non-fluorescence image such as a bright field image, a phase difference image, a phase image, and an in-line hologram image. For example, as a method for acquiring a non-fluorescence image, various observation methods such as a Schlieren method, a phase difference contrast method, a polarization observation method, and an epi-illumination method can be employed. The position of the illumination light source is not limited to below the stage 20, and may be above the stage 20 or around the objective lens 44. In addition, not only a method of performing focus control in real time, but also another method such as a prefocus map method of recording focus coordinates (Z coordinates) in advance may be employed.

Note that, in the above description, the line illumination as the excitation light includes two line illuminations Ex1 and Ex2 but is not limited thereto, and may be three, four, or five or more. In addition, each line illumination may include a plurality of excitation wavelengths selected so that the color separation performance is not degraded as much as possible. Further, even if there is one line illumination, if it is an excitation light source including a plurality of excitation wavelengths and each excitation wavelength is recorded in association with the data acquired by the imaging element 32, it is possible to obtain a polychromatic spectrum although it is not possible to obtain separability to be parallel to different axes.

The application example in which the technology according to the present disclosure is applied to the fluorescence observation apparatus 500 has been described above. Note that the above-described configuration described with reference to FIGS. 35 and 36 is merely an example, and the configuration of the fluorescence observation apparatus 500 according to the present embodiment is not limited to such an example. For example, the fluorescence observation apparatus 500 may not necessarily include all of the configurations shown in FIGS. 35 and 36, and may include a configuration not shown in FIGS. 35 and 36.

### 4. APPLICATION EXAMPLE

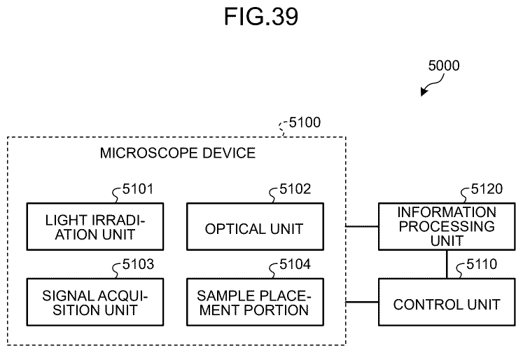

The technology according to the present disclosure can be applied to, for example, a microscope system and the like. Hereinafter, a configuration example of a microscope system 5000 that can be applied will be described with reference to FIGS. 39 to 41. A microscope device 5100 which is a part of the microscope system 5000 functions as an imaging device.

FIG. 39 shows an example configuration of a microscope system of the present disclosure. A microscope system 5000 shown in FIG. 39 includes a microscope device 5100, a control unit 5110, and an information processing unit 5120. The microscope device 5100 includes a light irradiation unit 5101, an optical unit 5102, and a signal acquisition unit 5103. The microscope device 5100 may further include a sample placement portion 5104 on which a biological sample S is placed. Note that the configuration of the microscope device is not limited to that shown in FIG. 39. For example, the light irradiation unit 5101 may exist outside the microscope device 5100, and a light source not included in the microscope device 5100 may be used as the light irradiation unit 5101. Alternatively, the light irradiation unit 5101 may be disposed so that the sample placement portion 5104 is sandwiched between the light irradiation unit 5101 and the optical unit 5102, and may be disposed on the side at which the optical unit 5102 exists, for example. The microscope device 5100 may be designed to be capable of performing one or more of the following: bright-field observation, phase contrast observation, differential interference contrast observation, polarization observation, fluorescent observation, and darkfield observation.

The microscope system 5000 may be designed as a so-called whole slide imaging (WSI) system or a digital pathology imaging system, and can be used for pathological diagnosis. Alternatively, the microscope system 5000 may be designed as a fluorescence imaging system, or particularly, as a multiple fluorescence imaging system.

For example, the microscope system 5000 may be used to make an intraoperative pathological diagnosis or a telepathological diagnosis. In the intraoperative pathological diagnosis, the microscope device 5100 can acquire the data of the biological sample S acquired from the subject of the operation while the operation is being performed, and then transmit the data to the information processing unit 5120. In the telepathological diagnosis, the microscope device 5100 can transmit the acquired data of the biological sample S to the information processing unit 5120 located in a place away from the microscope device 5100 (such as in another room or building). In these diagnoses, the information processing unit 5120 then receives and outputs the data. On the basis of the output data, the user of the information processing unit 5120 can make a pathological diagnosis.

### Biological Sample

The biological sample S may be a sample containing a biological component. The biological component may be a tissue, a cell, a liquid component of the living body (blood, urine, or the like), a culture, or a living cell (a myocardial cell, a nerve cell, a fertilized egg, or the like).

The biological sample may be a solid, or may be a specimen fixed with a fixing reagent such as paraffin or a solid formed by freezing. The biological sample can be a section of the solid. A specific example of the biological sample may be a section of a biopsy sample.

The biological sample may be one that has been subjected to a treatment such as staining or labeling. The treatment may be staining for indicating the morphology of the biological component or for indicating the substance (surface antigen or the like) contained in the biological component, and can be hematoxylin-eosin (HE) staining or immunohistochemistry staining, for example. The biological sample may be one that has been subjected to the above treatment with one or more reagents, and the reagent(s) can be a fluorescent dye, a coloring reagent, a fluorescent protein, or a fluorescence-labeled antibody.

The specimen may be prepared from a tissue sample for the purpose of pathological diagnosis or clinical examination. Alternatively, the specimen is not necessarily of the human body, and may be derived from an animal, a plant, or some other material. The specimen may differ in property, depending on the type of the tissue being used (such as an organ or a cell, for example), the type of the disease being examined, the attributes of the subject (such as age, gender, blood type, and race, for example), or the subject's daily habits (such as an eating habit, an exercise habit, and a smoking habit, for example). The specimen may be accompanied by identification information (bar code, QR code (registered trademark), or the like) for identifying each specimen, and be managed in accordance with the identification information.

### Light Irradiation Unit

The light irradiation unit 5101 is a light source for illuminating the biological sample S, and is an optical unit that guides light emitted from the light source to a specimen. The light source can illuminate a biological sample with visible light, ultraviolet light, infrared light, or a combination thereof. The light source may be one or more of the following: a halogen light source, a laser light source, an LED light source, a mercury light source, and a xenon light source. The light source in fluorescent observation may be of a plurality of types and/or wavelengths, and the types and the wavelengths may be appropriately selected by a person skilled in the art. The light irradiation unit may have a configuration of a transmissive type, a reflective type, or an epi-illumination type (a coaxial epi-illumination type or a side-illumination type).

### Optical Unit

The optical unit 5102 is designed to guide the light from the biological sample S to the signal acquisition unit 5103. The optical unit may be designed to enable the microscope device 5100 to observe or capture an image of the biological sample S.

The optical unit 5102 may include an objective lens. The type of the objective lens may be appropriately selected by a person skilled in the art, in accordance with the observation method. The optical unit may also include a relay lens for relaying an image magnified by the objective lens to the signal acquisition unit. The optical unit may further include optical components other than the objective lens and the relay lens, and the optical components may be an eyepiece, a phase plate, a condenser lens, and the like.

The optical unit 5102 may further include a wavelength Separation unit designed to separate light having a predetermined wavelength from the light from the biological sample S. The wavelength separation unit may be designed to selectively cause light having a predetermined wavelength or a predetermined wavelength range to reach the signal acquisition unit. The wavelength separation unit may include one or more of the following: a filter, a polarizing plate, a prism (Wollaston prism), and a diffraction grating that selectively pass light, for example. The optical component(s) included in the wavelength separation unit may be disposed in the optical path from the objective lens to the signal acquisition unit, for example. The wavelength separation unit is provided in the microscope device in a case where fluorescent observation is performed, or particularly, where an excitation light irradiation unit is included. The wavelength separation unit may be designed to separate fluorescence or white light from fluorescence.

### Signal Acquisition Unit

The signal acquisition unit 5103 may be designed to receive light from the biological sample S, and convert the light into an electrical signal, or particularly, into a digital electrical signal. The signal acquisition unit may be designed to be capable of acquiring data about the biological sample S, on the basis of the electrical signal. The signal acquisition unit may be designed to be capable of acquiring data of an image (a captured image, or particularly, a still image, a time-lapse image, or a moving image) of the biological sample S, or particularly, may be designed to acquire data of an image enlarged by the optical unit. The signal acquisition unit includes one or more image sensors, CMOSs, CCDs, or the like that include a plurality of pixels arranged in one-or two-dimensional manner. The signal acquisition unit may include an image sensor for acquiring a low-resolution image and an image sensor for acquiring a high-resolution image, or may include an image sensor for sensing for AF or the like and an image sensor for outputting an image for observation or the like. The image sensor may include not only the plurality of pixels, but also a signal processing unit (including one or more of the following: a CPU, a DSP, and a memory) that performs signal processing using pixel signals from the respective pixels, and an output control unit that controls outputting of image data generated from the pixel signals and processed data generated by the signal processing unit. The image sensor including the plurality of pixels, the signal processing unit, and the output control unit can be preferably designed as a one-chip semiconductor device. Note that the microscope system 5000 may further include an event detection sensor. The event detection sensor includes a pixel that photoelectrically converts incident light, and may be designed to detect that a change in the luminance of the pixel exceeds a predetermined threshold, and regard the change as an event. The event detection sensor may be of an asynchronous type.

### Control Unit

The control unit 5110 controls imaging being performed by the microscope device 5100. For the imaging control, the control unit can drive movement of the optical unit 5102 and/or the sample placement portion 5104, to adjust the positional relationship between the optical unit and the sample placement portion. The control unit 5110 can move the optical unit and/or the sample placement portion in a direction toward or away from each other (in the optical axis direction of the objective lens, for example). The control unit may also move the optical unit and/or the sample placement portion in any direction in a plane perpendicular to the optical axis direction. For the imaging control, the control unit may control the light irradiation unit 5101 and/or the signal acquisition unit 5103.

### Sample Placement Portion

The sample placement portion 5104 may be designed to be capable of securing the position of a biological sample on the sample placement portion, and may be a so-called stage. The sample placement portion 5104 may be designed to be capable of moving the position of the biological sample in the optical axis direction of the objective lens and/or in a direction perpendicular to the optical axis direction.

### Information Processing Unit

The information processing unit 5120 can acquire, from the microscope device 5100, data (imaging data or the like) acquired by the microscope device 5100. The information processing unit can perform image processing on the imaging data. The image processing may include an unmixing process, or more specifically, a spectral unmixing process. The unmixing process may include a process of extracting data of the optical component of a predetermined wavelength or in a predetermined wavelength range from the imaging data to generate image data, or a process of removing data of the optical component of a predetermined wavelength or in a predetermined wavelength range from the imaging data. The image processing may also include an autofluorescence separation process for separating the autofluorescence component and the dye component of a tissue section, and a fluorescence separation process for separating wavelengths between dyes having different fluorescence wavelengths from each other. The autofluorescence separation process may include a process of removing the autofluorescence component from image information about another specimen, using an autofluorescence signal extracted from one specimen of the plurality of specimens having the same or similar properties.

The information processing unit 5120 may transmit data for the imaging control to the control unit 5110, and the control unit 5110 that has received the data may control the imaging being by the microscope device 5100 in accordance with the data.

The information processing unit 5120 may be designed as an information processing device such as a general-purpose computer, and may include a CPU, RAM, and ROM. The information processing unit may be included in the housing of the microscope device 5100, or may be located outside the housing. Further, the various processes or functions to be executed by the information processing unit may be realized by a server computer or a cloud connected via a network.

The method to be implemented by the microscope device 5100 to capture an image of the biological sample S may be appropriately selected by a person skilled in the art, in accordance with the type of the biological sample, the purpose of imaging, and the like. Examples of the imaging method are described below.

One example of the imaging method is as follows. The microscope device can first identify an imaging target region. The imaging target region may be identified so as to cover the entire region in which the biological sample exists, or may be identified so as to cover the target portion (the portion in which the target tissue section, the target cell, or the target lesion exists) of the biological sample. Next, the microscope device divides the imaging target region into a plurality of divided regions of a predetermined size, and the microscope device sequentially captures images of the respective divided regions. As a result, an image of each divided region is acquired.

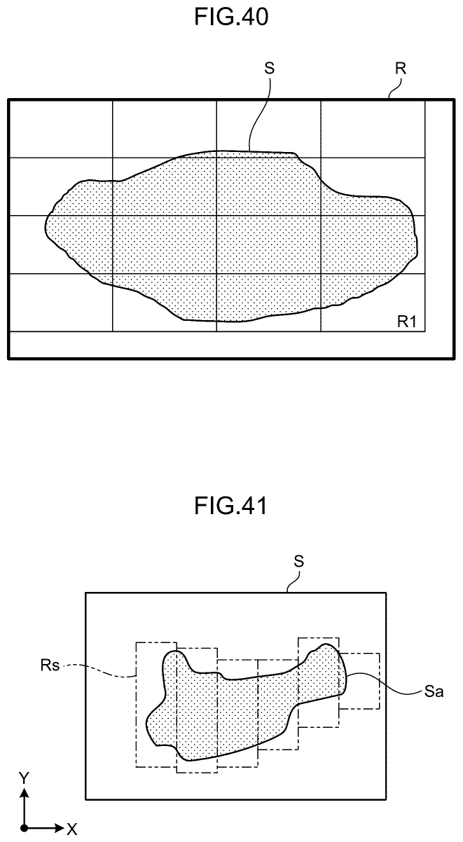

As shown in FIG. 40, the microscope device identifies an imaging target region R that covers the entire biological sample S. The microscope device then divides the imaging target region R into 16 divided regions. The microscope device then captures an image of a divided region R1, and next captures one of the regions included in the imaging target region R, such as an image of a region adjacent to the divided region R1. After that, divided region imaging is performed until images of all the divided regions have been captured. Note that an image of a region other than the imaging target region R may also be captured on the basis of captured image information about the divided regions.

The positional relationship between the microscope device and the sample placement portion is adjusted so that an image of the next divided region is captured after one divided region is captured. The adjustment may be performed by moving the microscope device, moving the sample placement portion, or moving both. In this example, the imaging device that captures an image of each divided region may be a two-dimensional image sensor (an area sensor) or a one-dimensional image sensor (a line sensor). The signal acquisition unit may capture an image of each divided region via the optical unit. Further, images of the respective divided regions may be continuously captured while the microscope device and/or the sample placement portion is moved, or movement of the microscope device and/or the sample placement portion may be stopped every time an image of a divided region is captured. The imaging target region may be divided so that the respective divided regions partially overlap, or the imaging target region may be divided so that the respective divided regions do not overlap. A plurality of images of each divided region may be captured while the imaging conditions such as the focal length and/or the exposure time are changed.

The information processing device can also generate image data of a wider region by stitching a plurality of adjacent divided regions. As the stitching process is performed on the entire imaging target region, an image of a wider region can be acquired with respect to the imaging target region. Also, image data with a lower resolution can be generated from the images of the divided regions or the images subjected to the stitching process.

Another example of the imaging method is as follows. The microscope device can first identify an imaging target region. The imaging target region may be identified so as to cover the entire region in which the biological sample exists, or may be identified so as to cover the target portion (the portion in which the target tissue section or the target cell exists) of the biological sample. Next, the microscope device scans a region (also referred to as a “divided scan region”) of the imaging target region in one direction (also referred to as a “scan direction”) in a plane perpendicular to the optical axis, and thus captures an image. After the scanning of the divided scan region is completed, the divided scan region next to the scan region is then scanned. These scanning operations are repeated until an image of the entire imaging target region is captured.

As shown in FIG. 41, the microscope device identifies a region (a gray portion) in which a tissue section of the biological sample S exists, as an imaging target region Sa. The microscope device then scans a divided scan region Rs of the imaging target region Sa in the Y-axis direction. After completing the scanning of the divided scan region Rs, the microscope device then scans the divided scan region that is the next in the X-axis direction. This operation is repeated until scanning of the entire imaging target region Sa is completed.

For the scanning of each divided scan region, the positional relationship between the microscope device and the sample placement portion is adjusted so that an image of the next divided scan region is captured after an image of one divided scan region is captured. The adjustment may be performed by moving the microscope device, moving the sample placement portion, or moving both. In this example, the imaging device that captures an image of each divided scan region may be a one-dimensional image sensor (a line sensor) or a two-dimensional image sensor (an area sensor). The signal acquisition unit may capture an image of each divided region via a magnifying optical system. Also, images of the respective divided scan regions may be continuously captured while the microscope device and/or the sample placement portion is moved. The imaging target region may be divided so that the respective divided scan regions partially overlap, or the imaging target region may be divided so that the respective divided scan regions do not overlap. A plurality of images of each divided scan region may be captured while the imaging conditions such as the focal length and/or the exposure time are changed.

The information processing device can also generate image data of a wider region by stitching a plurality of adjacent divided scan regions. As the stitching process is performed on the entire imaging target region, an image of a wider region can be acquired with respect to the imaging target region. Also, image data with a lower resolution can be generated from the images of the divided scan regions or the images subjected to the stitching process.

### 5. CONFIGURATION EXAMPLE OF HARDWARE

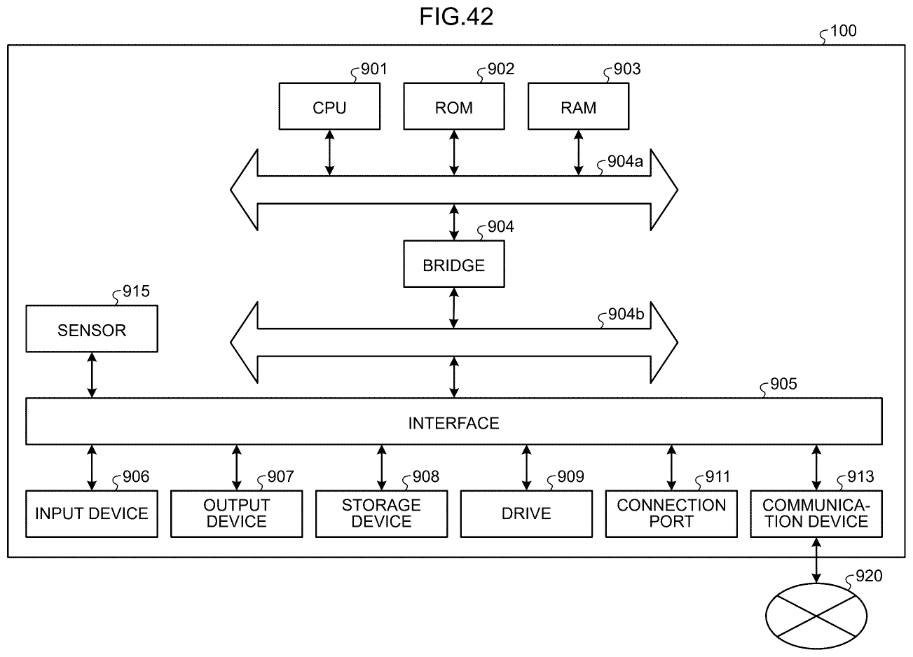

A hardware configuration example of the information processing device 100 according to each embodiment (or each modification) will be described with reference to FIG. 42. FIG. 42 is a block diagram showing an example of a schematic configuration of hardware of the information processing device 100. Various processes by the information processing device 100 are implemented, for example, by cooperation of software and hardware described below.

As shown in FIG. 42, the information processing device 100 includes a central processing unit (CPU) 901, a read only memory (ROM) 902, a random access memory (RAM) 903, and a host bus 904a. Furthermore, the information processing device 100 includes a bridge 904, an external bus 904b, an interface 905, an input device 906, an output device 907, a storage device 908, a drive 909, a connection port 911, a communication device 913, and a sensor 915. The information processing device 100 may include a processing circuit such as a DSP or an ASIC instead of or in addition to the CPU 901.

The CPU 901 functions as an arithmetic processing device and a control device, and controls the overall operation in the information processing device 100 according to various programs. In addition, the CPU 901 may be a microprocessor. The ROM 902 stores programs, operation parameters, and the like used by the CPU 901. The RAM 903 primarily stores programs used in the execution of the CPU 901, parameters that appropriately change in the execution, and the like. The CPU 901 can embody, for example, at least the processing unit 130 and the control unit 150 of the information processing device 100.

The CPU 901, the ROM 902, and the RAM 903 are mutually connected by a host bus 904a including a CPU bus and the like. The host bus 904a is connected to the external bus 904b such as a peripheral component interconnect/interface (PCI) bus via the bridge 904. Note that the host bus 904a, the bridge 904, and the external bus 904b do not necessarily need to be configured separately, and these functions may be mounted on one bus.

The input device 906 is implemented by, for example, a device to which information is input by an implementer, such as a mouse, a keyboard, a touch panel, a button, a microphone, a switch, and a lever. Furthermore, the input device 906 may be, for example, a remote control device using infrared rays or other radio waves, or may be an external connection device such as a mobile phone or a PDA corresponding to the operation of the information processing device 100. Furthermore, the input device 906 may include, for example, an input control circuit that generates an input signal on the basis of information input by the implementer using the above input units and outputs the input signal to the CPU 901. By operating the input device 906, the implementer can input various data to the information processing device and instruct the information processing device 100 to perform a processing operation. The input device 906 can embody at least the operating unit 160 of the information processing device 100, for example.

The output device 907 is formed by a device capable of visually or audibly notifying the implementer of acquired information. Examples of such a device include a display device such as a CRT display device, a liquid crystal display device, a plasma display device, an EL display device, and a lamp, a sound output device such as a speaker and a headphone, and a printer device. The output device 907 can embody at least the display unit 140 of the information processing device 100, for example.

The storage device 908 is a device for storing data. The storage device 908 is achieved by, for example, a magnetic storage device such as an HDD, a semiconductor storage device, an optical storage device, a magneto-optical storage device, or the like. The storage device 908 may include a storage medium, a recording device that records data in the storage medium, a reading device that reads data from the storage medium, a deletion device that deletes data recorded in the storage medium, and the like. The storage device 908 stores programs and various data executed by the CPU 901, various data acquired from the outside, and the like. The storage device 908 can embody at least the storage unit 120 of the information processing device 100, for example.

The drive 909 is a reader/writer for a storage medium, and is built in or externally attached to the information processing device 100. The drive 909 reads information recorded in a removable storage medium such as a mounted magnetic disk, optical disk, magneto-optical disk, or semiconductor memory, and outputs the information to the RAM 903. Furthermore, the drive 909 can also write information to a removable storage medium.

The connection port 911 is an interface connected to an external device, and is a connection port to an external device capable of transmitting data by, for example, a universal serial bus (USB).

The communication device 913 is, for example, a communication interface formed by a communication device or the like for connecting to the network 920. The communication device 913 is, for example, a communication card for wired or wireless local area network (LAN), long term evolution (LTE), Bluetooth (registered trademark), wireless USB (WUSB), or the like. Furthermore, the communication device 913 may be a router for optical communication, a router for asymmetric digital subscriber line (ADSL), a modem for various types of communication, or the like. For example, the communication device 913 can transmit and receive signals and the like to and from the Internet and other communication devices according to a predetermined protocol such as TCP/IP.

In the present embodiment, the sensor 915 includes a sensor capable of acquiring a spectrum (for example, an imaging element or the like), but may include another sensor (for example, an acceleration sensor, a gyro sensor, a geomagnetic sensor, a pressure-sensitive sensor, a sound sensor, a distance measuring sensor, or the like). The sensor 915 can embody at least the image acquisition unit 112 of the information processing device 100, for example.

Note that the network 920 is a wired or wireless transmission path of information transmitted from a device connected to the network 920. For example, the network 920 may include a public network such as the Internet, a telephone network, or a satellite communication network, various local area networks (LANs) including Ethernet (registered trademark), a wide area network (WAN), or the like. In addition, the network 920 may include a dedicated line network such as an Internet protocol-virtual private network (IP-VPN).

The hardware configuration example capable of implementing the functions of the information processing device 100 has been described above. Each of the above-described components may be implemented using a general-purpose member, or may be implemented by hardware specialized for the function of each component. Therefore, it is possible to appropriately change the hardware configuration to be used according to the technical level at the time of implementing the present disclosure.

Note that a computer program for implementing each function of the information processing device 100 as described above can be created and mounted on a PC or the like. Furthermore, it is also possible to provide a computer-readable recording medium storing such a computer program. The recording medium is, for example, a magnetic disk, an optical disk, a magneto-optical disk, a flash memory, or the like. In addition, the computer program described above may be distributed via, for example, a network without using the recording medium.

### 6. APPENDIXES

Note that the present technique can also take the following configurations.
(1)

An information processing device comprising:
an acquisition unit that acquires, from a sample including a biological sample, a fluorescence spectrum deriving from the biological sample and positional information of the biological sample;
a specifying unit that specifies, from the fluorescence spectrum, pieces of information concerning a plurality of different biomarkers of the biological sample linked with the positional information of the biological sample;
a standardization unit that executes information complementation or exclusion on the pieces of information concerning the plurality of biomarkers; and
a correlation analysis unit that performs, on the pieces of information concerning the plurality of biomarkers for which the information complementation or exclusion has been performed, matrix decomposition processing corresponding to a combination of the plurality of biomarkers and outputs a correlation among the pieces of information concerning the plurality of biomarkers.
(2)

The information processing device according to (1), wherein
the correlation analysis unit executes the matrix decomposition processing with joint non-negative matrix factorization (jNMF) on the pieces of information concerning the plurality of biomarkers for which the information complementation or exclusion has been executed.
(3)

The information processing device according to (1) or (2), wherein
the standardization unit calculates an average value and a standard deviation of the pieces of information concerning the plurality of biomarkers, generates random numbers having a normal distribution satisfying the calculated average value and the calculated standard deviation, and generates information complementing the pieces of information concerning the plurality of biomarkers.
(4)

The information processing device according to (1) or (2), wherein
the standardization unit replicates a part of the pieces of information concerning the plurality of biomarkers and generates information complementing the pieces of information concerning the plurality of biomarkers.
(5)

The information processing device according to any one of (1) to (4), wherein
the standardization unit excludes, from the pieces of information concerning the plurality of biomarkers, one or both of information concerning a region without a tissue and information concerning a region where a luminance value is equal to or larger than a predetermined threshold.
(6)

The information processing device according to any one of (1) to (5), further comprising
sorting unit that changes, based on arrangement order of a plurality of pieces of unit information included in information concerning one biomarker among the pieces of information concerning the plurality of biomarkers for which the information complementation or exclusion has been executed, arrangement order of a plurality of pieces of unit information included in information concerning other biomarkers, wherein
the correlation analysis unit performs the matrix decomposition processing on the pieces of information concerning the plurality of biomarkers, the arrangement order of which has been changed.
(7)

The information processing device according to any one of (1) to (6), wherein
the standardization unit calculates a coincidence degree between the pieces of information concerning the plurality of biomarkers and the pieces of information concerning the plurality of biomarkers for which the information complementation or exclusion has been performed.
(8)

The information processing device according to (7), further comprising a display processing unit that generates image information including the calculated coincidence degree.
(9)

The information processing device according to (7) or (8), wherein
the standardization unit repeats the information complementation or exclusion according to the calculated coincidence degree.
(10)

The information processing device according to any one of (1) to (9), further comprising
a display processing unit that generates image information including the correlation among the pieces of information concerning the plurality of biomarkers.
(11)

The information processing device according to (10), wherein
when the complementation of the information is executed, the display processing unit generates the image information to group and display the complemented information and actual information closest to the complemented information.
(12)

The information processing device according to (10) or (11), wherein
when the complementation of the information is executed, the display processing unit generates the image information to highlight the complemented information.
(13)

The information processing device according to any one of (10) to (12), wherein
when the complemented information is selected according to information selection for the image information, the display processing unit generates error image information indicating that the complemented information is selected.
(14)

The information processing device according to any one of (10) to (13), wherein
the display processing unit generates, according to information selection for the image information, image information concerning actual information closest to the complemented information.
(15)

The information processing device according to any one of (10) to (14), wherein
when the complementation of the information is executed, the display processing unit generates the image information not including the complemented information.
(16)

The information processing device according to any one of (1) to (15), further comprising
a selection unit that determines a predetermined region of the sample, wherein
the specifying unit specifies, from the fluorescence spectrum of the predetermined region, pieces of information concerning the plurality of biomarkers linked with the positional information of the biological sample in the predetermined region.
(17)

The information processing device according to (16), wherein
the predetermined region is set by a user.
(18)

The information processing device according to (16) or (17), wherein
the selection unit determines individual predetermined regions of a plurality of the samples,
the acquisition unit acquires the fluorescence spectrum and the positional information of the biological sample for each of the predetermined regions,
the specifying unit specifies, from the fluorescence spectrum for each of the predetermined regions, pieces of information concerning the plurality of biomarkers for each of the predetermined regions linked with the positional information of the biological sample for each of the predetermined regions,
the standardization unit executes information complementation or exclusion on the pieces of information concerning the plurality of biomarkers for each of the predetermined regions, and
the correlation analysis unit performs the matrix decomposition processing on the pieces of information concerning the plurality of biomarkers for each of the predetermined regions for which the information complementation or exclusion has been performed.
(19)

A biological sample analysis system comprising:
an imaging device that acquires a specimen image of a sample including a biological sample; and
an information processing device that processes the specimen image, wherein
the information processing device includes:
an acquisition unit that acquires, from the specimen image, a fluorescence spectrum deriving from the biological sample and positional information of the biological sample;
a specifying unit that specifies, from the fluorescence spectrum, pieces of information concerning a plurality of different biomarkers of the biological sample linked with the positional information of the biological sample;
a standardization unit that executes information complementation or exclusion on the pieces of information concerning the plurality of biomarkers; and
a correlation analysis unit that performs, on the pieces of information concerning the plurality of biomarkers for which the information complementation or exclusion has been performed, matrix decomposition processing corresponding to a combination of the plurality of biomarkers and outputs a correlation among the pieces of information concerning the plurality of biomarkers.
(20)

A biological sample analysis method comprising:
acquiring, from a sample including a biological sample, a fluorescence spectrum deriving from the biological sample and positional information of the biological sample;
specifying, from the fluorescence spectrum, pieces of information concerning a plurality of different biomarkers of the biological sample linked with the positional information of the biological sample;
executing information complementation or exclusion on the pieces of information concerning the plurality of biomarkers; and
performing, on the pieces of information concerning the plurality of biomarkers for which the information complementation or exclusion has been performed, matrix decomposition processing corresponding to a combination of the plurality of biomarkers and outputting a correlation among the pieces of information concerning the plurality of biomarkers.
(21)

A biological sample analysis system including the information processing device described in any one of (1) to (18).
(22)

A biological sample analysis method for performing analysis with the information processing device described in any one of (1) to (18).

### REFERENCE SIGNS LIST

1 OBSERVATION UNIT
2 PROCESSING UNIT
3 DISPLAY UNIT
10 EXCITATION UNIT
10A FLUORESCENCE REAGENT
11A REAGENT IDENTIFICATION INFORMATION
20 STAGE
20A SPECIMEN
21 STORAGE UNIT
21A SPECIMEN IDENTIFICATION INFORMATION
22 DATA CALIBRATION UNIT
23 IMAGE FORMING UNIT
30 SPECTROSCOPIC IMAGING UNIT
30A FLUORESCENCE STAINED SPECIMEN
40 OBSERVATION OPTICAL SYSTEM
50 SCANNING MECHANISM
60 FOCUS MECHANISM
70 NON-FLUORESCENCE OBSERVATION UNIT
80 CONTROL UNIT
100 INFORMATION PROCESSING DEVICE
110 ACQUISITION UNIT
111 INFORMATION ACQUISITION UNIT
112 IMAGE ACQUISITION UNIT
120 SAVING UNIT
121 INFORMATION SAVING UNIT
122 IMAGE INFORMATION SAVING UNIT
123 ANALYSIS RESULT SAVING UNIT
130 PROCESSING UNIT
131 ANALYSIS UNIT
132 IMAGE GENERATION UNIT
133 SPACE ANALYSIS UNIT
133A STANDARDIZATION UNIT
133a SELECTION UNIT
133b SPECIFYING UNIT
133c SORTING UNIT
133d CORRELATION ANALYSIS UNIT
133e ESTIMATION UNIT
134 DISPLAY PROCESSING UNIT
140 DISPLAY UNIT
150 CONTROL UNIT
160 OPERATION UNIT
200 DATABASE
500 FLUORESCENCE OBSERVATION DEVICE
5000 MICROSCOPE SYSTEM
5100 MICROSCOPE DEVICE
5101 LIGHT IRRADIATION UNIT
5102 OPTICAL UNIT
5103 SIGNAL ACQUISITION UNIT
5104 SAMPLE PLACEMENT PORTION
5110 CONTROL UNIT
5120 INFORMATION PROCESSING UNIT

1. An information processing device comprising:
an acquisition unit that acquires, from a sample including a biological sample, a fluorescence spectrum deriving from the biological sample and positional information of the biological sample;
a specifying unit that specifies, from the fluorescence spectrum, pieces of information concerning a plurality of different biomarkers of the biological sample linked with the positional information of the biological sample;
a standardization unit that executes information complementation or exclusion on the pieces of information concerning the plurality of biomarkers; and
a correlation analysis unit that performs, on the pieces of information concerning the plurality of biomarkers for which the information complementation or exclusion has been performed, matrix decomposition processing corresponding to a combination of the plurality of biomarkers and outputs a correlation among the pieces of information concerning the plurality of biomarkers.

2. The information processing device according to claim 1, wherein
the correlation analysis unit executes the matrix decomposition processing with joint non-negative matrix factorization (jNMF) on the pieces of information concerning the plurality of biomarkers for which the information complementation or exclusion has been executed.

3. The information processing device according to claim 1, wherein
the standardization unit calculates an average value and a standard deviation of the pieces of information concerning the plurality of biomarkers, generates random numbers having a normal distribution satisfying the calculated average value and the calculated standard deviation, and generates information complementing the pieces of information concerning the plurality of biomarkers.

4. The information processing device according to claim 1, wherein
the standardization unit replicates a part of the pieces of information concerning the plurality of biomarkers and generates information complementing the pieces of information concerning the plurality of biomarkers.

5. The information processing device according to claim 1, wherein
the standardization unit excludes, from the pieces of information concerning the plurality of biomarkers, one or both of information concerning a region without a tissue and information concerning a region where a luminance value is equal to or larger than a predetermined threshold.

6. The information processing device according to claim 1, further comprising
sorting unit that changes, based on arrangement order of a plurality of pieces of unit information included in information concerning one biomarker among the pieces of information concerning the plurality of biomarkers for which the information complementation or exclusion has been executed, arrangement order of a plurality of pieces of unit information included in information concerning other biomarkers, wherein
the correlation analysis unit performs the matrix decomposition processing on the pieces of information concerning the plurality of biomarkers, the arrangement order of which has been changed.

7. The information processing device according to claim 1, wherein
the standardization unit calculates a coincidence degree between the pieces of information concerning the plurality of biomarkers and the pieces of information concerning the plurality of biomarkers for which the information complementation or exclusion has been performed.

8. The information processing device according to claim 7, further comprising a display processing unit that generates image information including the calculated coincidence degree.

9. The information processing device according to claim 7, wherein
the standardization unit repeats the information complementation or exclusion according to the calculated coincidence degree.

10. The information processing device according to claim 1, further comprising
a display processing unit that generates image information including the correlation among the pieces of information concerning the plurality of biomarkers.

11. The information processing device according to claim 10, wherein
when the complementation of the information is executed, the display processing unit generates the image information to group and display the complemented information and actual information closest to the complemented information.

12. The information processing device according to claim 10, wherein
when the complementation of the information is executed, the display processing unit generates the image information to highlight the complemented information.

13. The information processing device according to claim 10, wherein
when the complemented information is selected according to information selection for the image information, the display processing unit generates error image information indicating that the complemented information is selected.

14. The information processing device according to claim 10, wherein
the display processing unit generates, according to information selection for the image information, image information concerning actual information closest to the complemented information.

15. The information processing device according to claim 10, wherein
when the complementation of the information is executed, the display processing unit generates the image information not including the complemented information.

16. The information processing device according to claim 1, further comprising
a selection unit that determines a predetermined region of the sample, wherein
the specifying unit specifies, from the fluorescence spectrum of the predetermined region, pieces of information concerning the plurality of biomarkers linked with the positional information of the biological sample in the predetermined region.

17. The information processing device according to claim 16, wherein
the predetermined region is set by a user.

18. The information processing device according to claim 16, wherein
the selection unit determines individual predetermined regions of a plurality of the samples,
the acquisition unit acquires the fluorescence spectrum and the positional information of the biological sample for each of the predetermined regions,
the specifying unit specifies, from the fluorescence spectrum for each of the predetermined regions, pieces of information concerning the plurality of biomarkers for each of the predetermined regions linked with the positional information of the biological sample for each of the predetermined regions,
the standardization unit executes information complementation or exclusion on the pieces of information concerning the plurality of biomarkers for each of the predetermined regions, and
the correlation analysis unit performs the matrix decomposition processing on the pieces of information concerning the plurality of biomarkers for each of the predetermined regions for which the information complementation or exclusion has been performed.

19. A biological sample analysis system comprising:
an imaging device that acquires a specimen image of a sample including a biological sample; and
an information processing device that processes the specimen image, wherein
the information processing device includes:
an acquisition unit that acquires, from the specimen image, a fluorescence spectrum deriving from the biological sample and positional information of the biological sample;
a specifying unit that specifies, from the fluorescence spectrum, pieces of information concerning a plurality of different biomarkers of the biological sample linked with the positional information of the biological sample;
a standardization unit that executes information complementation or exclusion on the pieces of information concerning the plurality of biomarkers; and
a correlation analysis unit that performs, on the pieces of information concerning the plurality of biomarkers for which the information complementation or exclusion has been performed, matrix decomposition processing corresponding to a combination of the plurality of biomarkers and outputs a correlation among the pieces of information concerning the plurality of biomarkers.

20. A biological sample analysis method comprising:
acquiring, from a sample including a biological sample, a fluorescence spectrum deriving from the biological sample and positional information of the biological sample;
specifying, from the fluorescence spectrum, pieces of information concerning a plurality of different biomarkers of the biological sample linked with the positional information of the biological sample;
executing information complementation or exclusion on the pieces of information concerning the plurality of biomarkers; and
performing, on the pieces of information concerning the plurality of biomarkers for which the information complementation or exclusion has been performed, matrix decomposition processing corresponding to a combination of the plurality of biomarkers and outputting a correlation among the pieces of information concerning the plurality of biomarkers.
